(19) United States
(12) Patent Application Publication
(10) Pub. No.: US 2026/0053949 A1
(43) Pub. Date: Feb. 26, 2026

(54) HYBRID AAV CAPSID AND USES THEREOF

(71) Applicant: AAV Gene Therapeutics, Inc., Beverly, MA (US)

(72) Inventor: Senthil Ramu, Boston, MA (US)

(21) Appl. No.: 19/105,382

(22) PCT Filed: Sep. 1, 2023
(86) PCT No.: PCT/US2023/031798

§ 371 Date: Feb. 21, 2025

Related U.S. Application Data
(60) Provisional application No. 63/403,066, filed on Sep. 1, 2022.

(51) Int. Cl. A61K 48/00; C07K 14/005; C12N 15/86

(52) U.S. Cl. CPC A61K 48/0041; C07K 14/005; C12N 15/86; C07K 2319/09; C12N 2750/14122; C12N 2750/14143; C12N 2750/14152; C12N 2800/22; C12N 2830/50

(57) ABSTRACT

The invention described herein provides an engineered hybrid capsid for adeno-associated virus (AAV), which capsid is a chimeric fusion between two different clinically validated AAV capsids with different AAV serotypes for enhanced nuclear delivery (e.g., AAV 8 and AAV9). Compositions and methods of use thereof are also provided.

### REFERENCE TO RELATED APPLICATION

This application claims the benefit of the filing date and priority to U.S. Provisional Patent Application No. 63/403,066, filed on Sep. 1, 2022, the entire contents of which including any drawings and sequences are incorporated herein by reference.

### BACKGROUND OF THE INVENTION

AAV is a non-pathogenic virus belonging to the genus Dependoparvovims within the family Parvoviridae. AAV is a non-enveloped virus composed of a capsid of about 26 nm in diameter and a single-stranded DNA genome of 4.7 kb. The AAV genome contains only two genes, rep and cap, flanked by two palindromic Inverted terminal Repeats (ITR) sequences that serve as the viral origins of replication and the packaging signal.

The AAV cap gene encodes three structural proteins—VP1, VP2 and VP3—using overlapping coding sequences and alternative start codons. VP1 is the longest translated protein, followed by VP2 and VP3. These three structural proteins share the same C-terminal end, which is all of VP3. The AAV viral capsid or viral particle consists of all three capsid proteins at a ratio of 1:1:10 (VP1:VP2:VP3). VP3 is the main structural component that builds the particle.

Using the widely studied AAV2 has an example and reference, VP1 has a 735 amino acids (GenBank accession number YP_680426.1). AAV2 VP2 (598 amino acids) starts at Threonine 138 (T138) of VP1. AAV2 VP3 (533 amino acids) starts at the methionine 203 (M203) of VP1.

The rep gene encodes four proteins required for viral replication—Rep78, Rep68, Rep52 and Rep40.

Recombinant AAV (rAAV) vectors encapsidate an ITR-flanked rAAV genome in which a gene expression cassette for a gene of interest (GOI) replaces the AAV protein-coding genes rep and cap.

Since naturally existing AAV capsids have limited host cell tropism, there is a need for novel AAV capsids that at least partially overcome such limitations for expanded therapeutic needs.

### SUMMARY OF THE INVENTION

One aspect of the invention provides a polynucleotide encoding an engineered adeno-associated virus (AAV) capsid comprising a viral protein 1 (VP1), a viral protein 2 (VP2), and a viral protein 3 (VP3), wherein said VP1 comprises: (a) a first portion from a first AAV capsid (e.g., AAV8) VP1, wherein said first portion comprises a nuclear localization sequence (NLS) of said first AAV capsid (e.g., AAV8) VP1; and, (b) a second portion from a second AAV capsid (e.g., AAV9) VP1, wherein said second portion comprises a tropism region associated with a tropism of the second capsid (e.g., AAV9) VP1 for a target cell; wherein the first and the second AAV capsids have different tropism or serotype.

In certain embodiments, the NLS, or said first portion comprising said NLS, when present in a fusion with a reporter (e.g., a fluorescent protein such as GFP), directs the subcellular localization of the fusion to the nucleus of a cell (e.g., muscle cell or myoblast) expressing the fusion.

In certain embodiments, the first AAV capsid is from a clinically validated AAV serotype (such as AAV5, AAV6, AAV8, or AAV9).

In certain embodiments, the first portion comprises the VP1-N region and the VP2-N region of the first AAV capsid VP1, and wherein the tropism region is within the VP3 region of the second AAV capsid VP1.

In certain embodiments, the first portion (of the first AAV capsid VP1) substantially excludes the VP3 region of said first AAV capsid VP1, and the second portion (of the second AAV capsid VP1) comprises, consists essentially of, or consists of the VP3 region of said second AAV capsid VP1.

In certain embodiments, the first AAV capsid VP1 is from AAV8, and said second AAV capsid VP1 is from AAV9.

In certain embodiments, the polynucleotide encodes the N-terminal 212-220 residues of AAV8 VP1.

In certain embodiments, the polynucleotide encodes the C-terminal 517-525 residues (e.g., residues 220-736 or residues 212-736) of AAV9 VP1.

In certain embodiments, the engineered AAV capsid comprises, consists essentially of, or consists of the polypeptide sequence of SEQ ID NO: 2, or a variant at least 90%, 91%, 92%, 93%, 94%, 95%, 96%, 97%, 98%, 99%, 99.2%, 99.4%, 99.6, 99.8%, or 99.9% identical thereto.

In certain embodiments, the polynucleotide is codon optimized for expression in a mammalian cell (such as HEK293T cells) or an insect cell (such as Sf9).

In certain embodiments, the polynucleotide comprises, consists essentially of, or consists of the polynucleotide sequence of SEQ ID NO: 1.

Another aspect of the invention provides a vector comprising the polynucleotide of the invention.

In certain embodiments, the vector comprises a promoter operably linked to a transcription cassette comprising the polynucleotide of the invention.

In certain embodiments, the vector further comprises a coding sequence for an AAV rep.

Another aspect of the invention provides an engineered adeno-associated virus (AAV) capsid VP1, VP2, and/or VP3, encoded by the polynucleotide of the invention.

Another aspect of the invention provides a host cell comprising the vector of the invention.

In certain embodiments, the host cell further comprises a gene-of-interest (GOI) flanked by an AAV ITR sequence, capable of being encapsidated in the capsid of the invention.

In certain embodiments, the GOI is operably linked to a promoter.

In certain embodiments, the promoter comprises: (1) a constitutively active eukaryotic promoter, (2) a muscle-specific promoter, a CNS-specific promoter, and/or (3) a synthetic promoter.

In certain embodiments, the GOI further encodes a 5′-UTR region, a 3′-UTR region, and/or a polyA signal.

Another aspect of the invention provides an AAV viral particle comprising the AAV capsid of the invention.

In certain embodiments, the AAV viral particle comprises an AAV vector genome comprising a GOI flanked by an AAV ITR sequence, wherein the GOI is operably linked to a promoter, optionally, the promoter comprises: (1) a constitutively active eukaryotic promoter, (2) a muscle-specific promoter, a CNS-specific Promoter, and/or (3) a synthetic promoter, and/or, optionally, the GOI further encodes a 5′-UTR region, a 3′-UTR region, and/or a polyA signal.

In certain embodiments, the GOI encodes a functional protein (such as a therapeutic protein), a functional nucleic acid, and/or an antibody or a portion thereof (such as a heavy chain and/or a light chain of the antibody).

In certain embodiments, the functional protein comprises a CRISPR/Cas effector enzyme (such as Cas9, Cas12, or Cas13), and/or wherein the functional nucleic acid is a single guide RNA (sgRNA), siRNA, miRNA, shRNA, antisense RNA, ribozyme, or aptamer.

In certain embodiments, the functional protein comprises α-glucosidase alglucosidase alfa, α-galactosidase A (α-Gal A), beta-glucocerebrosidase, lysosomal enzyme iduronate-2-sulfatase, asfotase alfa, lysosomal acid lipase (LAL), sebelipase alfa, Iduronidase (IDUA), arylsulfatase B (ARSB), SMPD1, or Insulin.

In certain embodiments, the functional protein comprises or is encoded by: CLN3 (Neuro Ceroid-Lipofuscinosis); FUCA1 (Fucosidosis); GAN (Giant Axonal Neuropathy); GALC (Globoid cell leukodystrophy); Mucolipidiosis Type IV GALC; Mucolipidiosis Type IV MCOLN1; Neuronal Ceroid Lipofusinoses PPT1; Niemann-Pick Disease SMPD1; HEXB (Sandhoff Disease); SGSH (Sanfilippo syndrome); HEXA (Tay-Sachs Disease); NEU1 (Sialidosis); SUMF1 (Multiple Sulfatase Deficiency); GAT1/SLCA1 (Childhood Epilepsy); CMT FIG. 4 (Peripheral Neuropathy); CLN5 (Neuronal Ceroid Lipofusinoses); AGA (Aspartylglycosaminuria); GDNF (Parkinsons Disease/Symptoms); GLB1 (GM1-Gangliosidosis); PMP22/MFN2 (Charcot-Marie-Tooth Type 1A); MECP2 (Retts Syndrome); LAMP2 (Dannon Disease); NAGLU (Mucopolysaccharidoses); GUSB (Sly Syndrome); SLC19A3 (Biotin basal ganglia disease); PLP1 (Pelizaeus-Merzbacher disease); TPP1/CLN2 (Neuronal Ceroid Lipofusinoses); ACY2/ASPA (Canavan Disease); MANBA (Beta-Mannosidosis); CTNS (Cystinosis); GNS (Mucopolysaccharidoses); HGSNAT (Mucopolysaccharidoses); SLC17A5 (Salla Disease); CLN6 (Jansky-Bielschowsky disease); CLN8 (Neuronal ceroid lipofuscinoses); GM2 gangliosidosis.

Another aspect of the invention provides a pharmaceutical composition comprising the engineered AAV capsid VP1 of the invention, or the AAV viral particle of the invention, and a pharmaceutically acceptable carrier or excipient.

Another aspect of the invention provides a method of treating a (genetic) disease or disorder in a subject, the method comprising introducing the AAV viral particle of the invention, or the pharmaceutical composition of the invention, into the subject.

In certain embodiments, the (genetic) disease or disorder is: (1) Pompe disease or GAA deficiency, and the GOI encodes α-glucosidase alglucosidase alfa; (2) Fabry disease or a deficiency of α-galactosidase A (α-Gal A), and the GOI encodes α-galactosidase A; (3) Gaucher disease or beta-glucocerebrosidase deficiency, and the GOI encodes beta-glucocerebrosidase; (4) Hunter syndrome or MPS-II, and the GOI encodes lysosomal enzyme iduronate-2-sulfatase; (5) hypophosphatasia (HPP), such as perinatal/infantile- and juvenile-onset HPP, and the GOI encodes asfotase alfa; (6) lysosomal acid lipase deficiency (LAL-D), and the GOI encodes lysosomal acid lipase (LAL) or sebelipase alfa; (7) Hurler syndrome/Scheie syndrome, and the GOI encodes Iduronidase (IDUA); (8) Maroteaux-Lamy syndrome, and the GOI encodes arylsulfatase B (ARSB); (9) sphingomyelinase deficiency (ASMD), and the GOI encodes SMPD1; or, (10) Type1 Diabetes, Type2 Diabetes and hyperglycemia, and the GOI encodes Insulin.

In certain embodiments, the (genetic) disease or disorder is: Neuro Ceroid-Lipofuscinosis; Fucosidosis; Giant Axonal Neuropathy; Globoid cell leukodystroph); Mucolipidiosis Type IV; Mucolipidiosis Type IV; Neuronal Ceroid Lipofusinoses; Niemann-Pick Disease; Sandhoff Disease; Sanfilippo syndrome; Tay-Sachs Disease; Sialidosis; Multiple Sulfatase Deficiency; Childhood Epilepsy; Peripheral Neuropathy; Neuronal Ceroid Lipofusinoses; Aspartylglycosaminuria; Parkinsons Disease/Symptoms; GM1-Gangliosidosis; Charcot-Marie-Tooth Type 1A; Retts Syndrome; Dannon Disease; Mucopolysaccharidoses; Sly Syndrome; Biotin basal ganglia disease; Pelizaeus-Merzbacher disease; Canavan Disease; Beta-Mannosidosis; Cystinosis; Mucopolysaccharidoses; Mucopolysaccharidoses; Salla Disease; Jansky-Bielschowsky disease; Neuronal ceroid lipofuscinoses; GM2 gangliosidosis.

Another aspect of the invention provides a method of producing the AAV viral particle of the invention, comprising introducing the polynucleotide of the invention into a packaging cell line that constitutively or inducibly expresses said polynucleotide and an AAV cap protein.

It should be understood that any one embodiment of any one of the aspects of the invention above, including specific embodiments described only in one section of the application, can be combined with one or more additional embodiments.

### BRIEF DESCRIPTION OF THE DRAWINGS

FIG. 1 is a schematic (not to scale) illustration of the conventional AAV delivery process from receptor-mediated endocytosis to nuclear transport. Typically, only about 20% of the cytoplasmic AAV viral particles enters target cell nucleus that results in transcription/translation of the GOI in the AAV viral vector, while the majority (about 80%) of the AAV viral particles are targeted to intracellular degradation through proteasome.

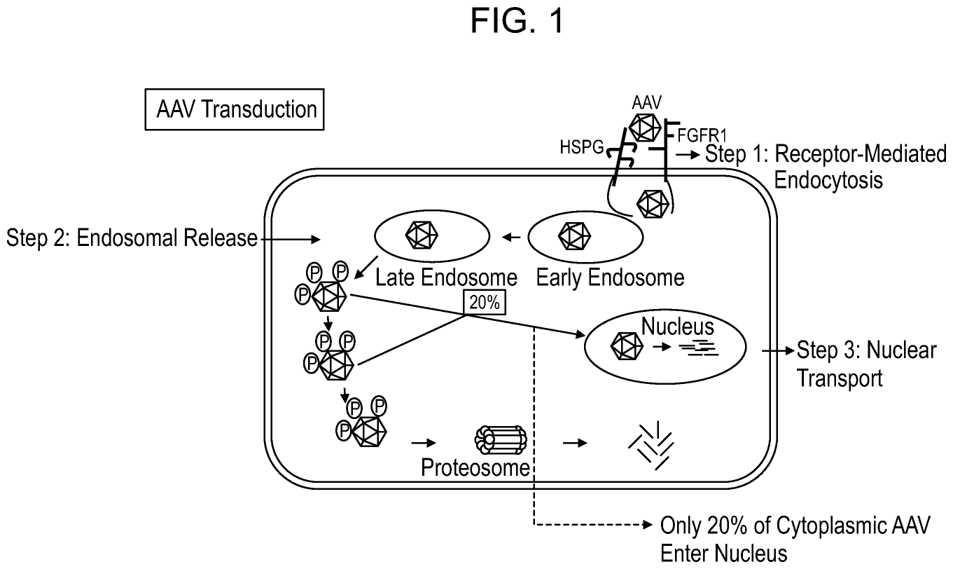

FIG. 2 is a schematic (not to scale) illustration showing the general scheme of the hybrid capsids of the invention. “VP1” stands for the VP1-N domain of the VP1 polypeptide. “VP2” stands for the VP2-N domain of the VP1 polypeptide. “VP3” stands for the VP3 domain of the VP1 polypeptide. The exact point of fusion between the two parental capsids are illustrated as, but are not necessarily at, the boundary of the VP2-N domain and the VP3 domain.

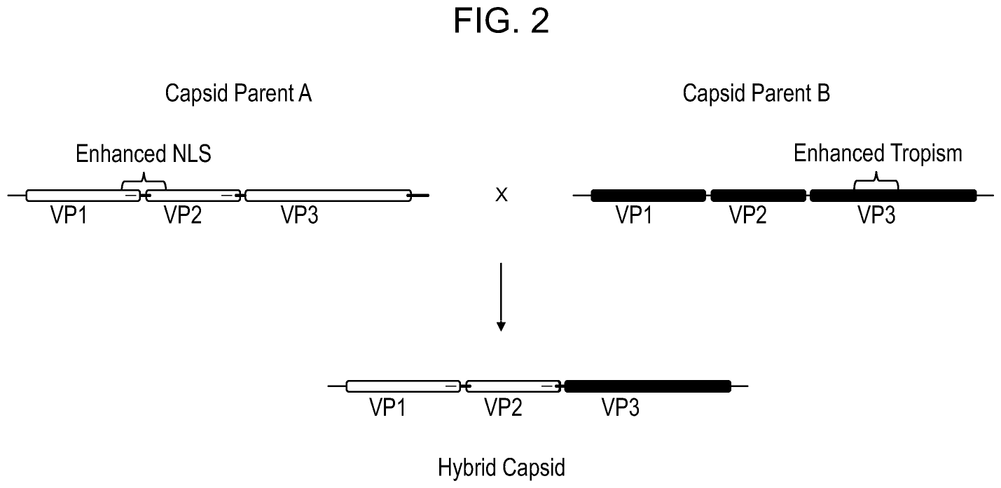

FIG. 3 is a schematic (not to scale) illustration showing several embodiments of the AAV genome fusion with a reporter (i.e., GFP in this case). Such constructs are useful for identifying parental capsids with enhanced NLS in the VP1-N and VP2-N domains.

FIG. 4 is a gel image showing representative AAV-GFP fusion constructs useful for testing enhanced nuclear delivery. (deleted a portion here). The NLS-GFP fusion can be used as a positive control. AAV5, 6, 8, and 9 have all been validated as being useful in human gene therapy clinical trials through i.v. dosing.

FIG. 5 shows the results of AAV-GFP fusion nuclear translocation analysis in muscle cells. DAPI staining (bottom panels) shows the location of the cell nucleus. GFP signals (top panels) overlapping/co-localizing with the DAPI signals signify expression of the reporter GFP inside the cell nucleus. AAV8 and 9 appeared to have stronger GFP expression in the nucleus compared to AAV5 and 6.

FIG. 6 shows the results of quantitative analysis of nuclear translocation for the various AAV-GFP fusions. AAV8 appeared to exhibit the strongest nuclear translocation among the tested capsids.

FIG. 7 shows the nuclear translocation intensity in muscle cells of the several tested AAV-GFP fusions.

FIG. 8 shows the results of quantitative analysis of nuclear translocation intensity for the various AAV-GFP fusions. AAV8 again appeared to exhibit the strongest nuclear translocation intensity among the tested capsids.

FIG. 9 shows a representative hybrid capsid (labeled in the figure as “AAV9-8”) of the invention comprising the VP1-N and VP2-N domains of AAV8 VP1, and the VP3 domain of the AAV9 VP1. Coding sequence for this hybrid capsid was cloned into an AAV repcap plasmid that expresses the AAV2 Rep and the AAV9-8 hybrid capsid, as verified by the gel image.

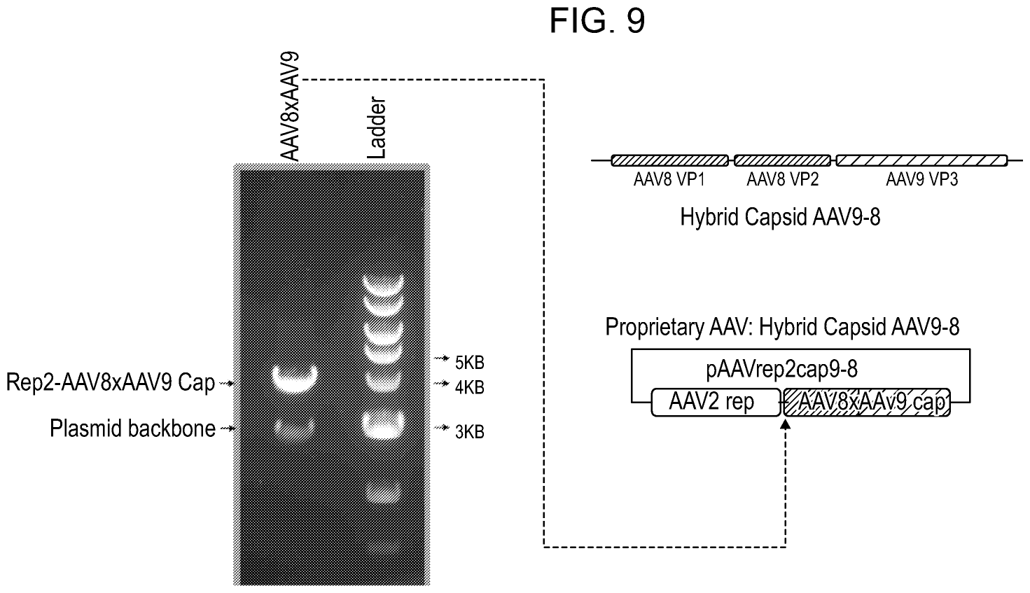

### DETAILED DESCRIPTION OF THE INVENTION

### 1. Overview

The AAV capsid protein can be referred to using several different numbering systems. For convenience, as used herein, the AAV protein sequences are referred to using VP1 numbering, which starts with amino acid 1 for the first residue of VP1.

The invention described herein provides, inter alia, an engineered or “hybrid” AAV capsid derived by fusion of AAV viral genomes from two different (preferably clinically validated) AAV serotypes. Such engineered capsids have enhanced nuclear translocation, due to the presence of a nuclear localization sequence (NLS) from a different serotype in the N-terminal portion (such as the VP1-N and/or VP2-N regions) of the hybrid capsid.

In other words, the invention described herein provides an engineered hybrid capsid for adeno-associated virus (AAV), which capsid is a cross or chimeric fusion between two different clinically validated AAV capsids, derived by fusing capsid parts from two different AAV serotypes. The subject capsid is created by genetic fusion between two parent capsids exhibiting superior genetic traits, wherein the first parent capsid harbors enhanced Nuclear Localization Signal (NLS), and the second parent capsid exhibits enhanced tropism.

Thus, in one aspect, the invention provides a polynucleotide encoding an engineered or hybrid adeno-associated virus (AAV) capsid comprising a viral protein 1 (VP1), a viral protein 2 (VP2), and a viral protein 3 (VP3), wherein said VP1 comprises: (a) a first portion from a first AAV capsid (e.g., AAV8) VP1, wherein said first portion comprises a nuclear localization sequence (NLS) of said first AAV capsid (e.g., AAV8) VP1; and, (b) a second portion from a second AAV capsid (e.g., AAV9) VP1, wherein said second portion comprises a tropism region associated with a tropism of the second capsid (e.g., AAV9) VP1 for a target cell; wherein the first and the second AAV capsids have different tropism or serotype.

In certain embodiments, the NLS, or the first portion comprising the NLS, when present in a fusion with a reporter (e.g., a fluorescent protein such as GFP), directs the subcellular localization of the fusion to the nucleus of a cell (e.g., muscle cell or myoblast) expressing the fusion.

For example, the presence of the NLS in the first portion can be tested or verified by creating a fusion protein between a putative NLS-containing sequence from the first AAV capsid, and a reporter such as a fluorescent protein (e.g., GFP). If the encoded fusion shows enhanced nuclear localization compared to the reporter alone control, upon transfection of a coding sequence for the fusion or the coding sequence for the reporter alone, the putative NLS-containing sequence from the first AAV capsid is verified to have a stronger Nuclear Localization Signal (NLS).

In certain embodiments, the NLS directs the subcellular localization of the fusion to the nucleus of a muscle cell or myoblast expressing the fusion. In certain embodiments, the muscle is a smooth muscle (e.g., diaphragm), a cardiac muscle (i.e. heart), or a skeletal muscle. In certain embodiments, the muscle cell is a myocyte, a myotube, a myoblast, or a satellite cell. In certain embodiments, the muscle is Tibialis (TA), Extensor Digitorum Longus (EDL), Quadriceps (Qua), Gastrocnemius (Ga), Soleus (Sol), Tricep, Bicep and/or Diaphragm.

In certain embodiments, the NLS directs the subcellular localization of the fusion to the nucleus of a neuronal cell in the CNS. In certain embodiments, the CNS includes brain, spinal cord, retina, optic nerve, and/or olfactory nerves and epithelium. In certain embodiments, the neuronal cell is a neuron, an astrocyte, a glial cell, an oligodendrocyte, an ependymal cell, or a microglia cell, etc.).

In certain embodiments, the first AAV capsid is from an (intravenously dosed) clinically validated AAV serotype. In certain embodiments, the second AAV capsid is from an (intravenously dosed) clinically validated AAV serotype. In certain embodiments, both the first and the second AAV capsids are from an (intravenously dosed) clinically validated AAV serotype.

In certain embodiments, the clinically validated AAV serotype includes AAV1, AAV2, AAV3, AAV4, AAV5, AAV6, AAV7, AAV8, AAV9, AAV2/3, AAV2/13, AAV-DJ, AAV-DJ8, AAV.PHP, AAV-PHP.B, AAV-PHP.EB, AAV2i8, AAV 2G9, AAV-Anc80, AAV-LK03, AAVcylO, AAVrh10, AAVrh32.33, AAVrh39, AAVrh43, AAVrh74, AAVHSC (such as AAVHSC7, AAVHSC15 and AAVHSC17), and AAV9.rh74-P1 (WO 2019/193119, incorporated by reference), porcine AAV (such as AAVpo1, AAVpo2.1, AAVpo4 and AAVpo6), and tyrosine, lysine and serine capsid mutants of AAV serotypes, or variants thereof.

In certain embodiments, the clinically validated AAV serotype includes AAV5, AAV6, AAV8, or AAV9.

In certain embodiments, the first portion comprises the VP1-N region and the VP2-N region of the first AAV capsid VP1 (and optionally the most N-terminal about 5, 10, 15, or 20 residues of the VP3 domain), and wherein the tropism region is within the VP3 region of the second AAV capsid VP1.

As used herein, the “VP1-N” region includes/refers to the N-terminal portion of a VP1 protein that is not present in VP2 and not present in VP3. This region is N-terminal to the first corresponding VP2 amino acid residue.

As used herein, the “VP2-N” region includes/refers to the N-terminal portion of a VP2 protein that is not present in VP3. The region is N-terminal to the first corresponding VP3 amino acid residue.

As used herein, the “VP3” domain includes/refers to the remaining portion of the VP1 protein that corresponds to the VP3 polypeptide. The VP3 domain of a VP1 polypeptide is C-terminal to the VP1-N and the VP2-N domains of the same VP1 polypeptide.

In certain embodiments, the first portion (of the first AAV capsid VP1) substantially excludes the VP3 domain of said first AAV capsid VP1. For example, the first portion may completely exclude the VP3 domain of the first AAV capsid VP1. In certain embodiments, however, the first portion may include a few residues or a small portion of the VP3 domain, such as 1, 2, 3, 4, 5, 6, 7, 8, 9, 10, 20, 25, 30, 25, 40, 45, or up to 50 residues of the VP3 domain. In certain embodiments, the first portion includes up to 1, 2, 3, 4, 5, 6, 7, 8, 9, 10, 11, 12, 13, 14, 15, 16, 17, 18, 19, or 20 residues of the VP3 domain.

In certain embodiments, the second portion (of the second AAV capsid VP1) comprises, consists essentially of, or consists of the VP3 domain of said second AAV capsid VP1. In certain embodiments, however, the second portion may include a few residues or a small portion of the VP2-N region sequence immediately N-terminal the VP3 domain, such as up to 1, 2, 3, 4, 5, 6, 7, 8, 9, 10, 20, 25, 30, 25, 40, 45, or up to 50 residues of the VP2-N domain immediately N-terminal to the VP3 domain.

In certain embodiments, the first AAV capsid VP1 is from AAV8.

In certain embodiments, the second AAV capsid VP1 is from AAV9.

In certain embodiments, the first AAV capsid VP1 is from AAV8, and the second AAV capsid VP1 is from AAV9.

In certain embodiments, the polynucleotide encodes the N-terminal 212-220 residues of AAV8 VP1 polypeptide (e.g., residues 1-212 or residues 1-220) of AAV8 VP1.

In certain embodiments, the polynucleotide encodes the C-terminal 517-525 residues of AAV9 VP1 polypeptide (e.g., residues 212-736 or 220-736) of AAV9 VP1.

In certain embodiments, the polynucleotide encodes residues 1-212 of AAV8 VP1 and encodes residues 212-736 of AAV9 VP1.

In certain embodiments, the polynucleotide encodes residues 1-220 of AAV8 VP1 and encodes residues 220-736 of AAV9 VP1.

In certain embodiments, the engineered AAV capsid comprises, consists essentially of, or consists of the polypeptide sequence of SEQ ID NO: 2, or a variant at least 90%, 91%, 92%, 93%, 94%, 95%, 96%, 97%, 98%, 99%, 99.2%, 99.4%, 99.6, 99.8%, or 99.9% identical thereto.

In certain embodiments, the polynucleotide is codon optimized for expression in a mammalian cell (such as HEK293T cells) or an insect cell (such as Sf9) that are suitable for AAV production as packaging cell lines.

In certain embodiments, the polynucleotide comprises, consists essentially of, or consists of the polynucleotide sequence of SEQ ID NO: 1.

In certain embodiments, the polynucleotide is DNA, RNA, or a synthetic or semi-synthetic nucleic acid.

In certain embodiments, upon infection a target host cell (e.g., muscle cell or neuronal cell), approximately 20%, 25%, 30%, 35%, 40%, 45%, 50% or more of the AAV vectors encapsidated in the subject capsid gain entry into nucleus.

While not wishing to be bound by any particular theory, it is believed that the hybrid capsid of the invention boosts expression of a gene of interest (GOI) in the host cell partly by boosting AAV vector encoding such GOI into the nucleus of the target host cell.

The engineered/hybrid AAV capsid protein of the invention is a functional AAV capsid which is able to form recombinant AAV viral particles which transduce a cell, tissue or organ, in particular a cell tissue or organ of interest (target cell, tissue or organ) and express a transgene in said cell, tissue or organ. Non-limiting cell, tissue or organ include muscle cells and neuronal tissues (including neurons and non-neuronal cells in the CNS (such as glial cells, astrocytes etc.)).

Furthermore, the engineered/hybrid AAV capsid protein has increased or improved nuclear localization compared to at least one of its parent AAV capsid protein(s).

The transgene expression levels achieved with the hybrid AAV capsid protein in the target cell, tissue or organ (e.g., muscle and neuronal cells) is advantageously at least of the same magnitude or significantly enhanced as that of a reference AAV serotype such as AAV9 for muscle and CNS tissues. While not wishing to be bound by any particular theory, it is believed that the enhanced delivery might be attributed to enhanced nuclear localization signal in hybrid capsid.

Another aspect of the invention provides a vector comprising the polynucleotide of the invention.

In certain embodiments, the vector comprises a promoter operably linked to a transcription cassette comprising the polynucleotide of the invention.

In certain embodiments, the vector further comprises a coding sequence for an AAV Rep protein (the rep/cap vector). The Rep protein may comprise Rep78 and/or Rep68, such as Rep78/Rep8 of AAV2.

Another aspect of the invention provides an engineered adeno-associated virus (AAV) capsid VP1, VP2, and/or VP3, encoded by the polynucleotide of the invention.

Another aspect of the invention provides a host cell comprising the vector of the invention.

In certain embodiments, the host cell further comprises a gene-of-interest (GOI) flanked by an AAV ITR sequence (such as AAV2 5′ and 3′ ITR sequences), capable of being encapsidated in the capsid of the invention.

In certain embodiments, the GOI is operably linked to a promoter.

In certain embodiments, the promoter comprises: (1) a constitutively active eukaryotic promoter, (2) a muscle-specific promoter, a CNS-specific promoter, and/or (3) a synthetic promoter.

In certain embodiments, the GOI further encodes a 5′-UTR region, a 3′-UTR region, and/or a polyA signal.

Another aspect of the invention provides an AAV viral particle comprising the AAV capsid of the invention.

In certain embodiments, the AAV viral particle comprises an AAV vector genome comprising a GOI flanked by an AAV ITR sequence, wherein the GOI is operably linked to a promoter. In certain embodiments, the promoter comprises: (1) a constitutively active eukaryotic promoter, (2) a muscle-specific promoter, a CNS-specific Promoter, and/or (3) a synthetic promoter. In certain embodiments, the GOI further encodes a 5′-UTR region, a 3′-UTR region, and/or a polyA signal.

In certain embodiments, the GOI encodes a functional protein (such as a therapeutic protein), a functional nucleic acid, and/or an antibody or a portion thereof (such as a heavy chain and/or a light chain of the antibody).

In certain embodiments, the functional protein comprises a CRISPR/Cas effector enzyme (such as Cas9, Cas12, or Cas13), and/or wherein the functional nucleic acid is a single guide RNA (sgRNA), siRNA, miRNA, shRNA, antisense RNA, ribozyme, or aptamer.

In certain embodiments, the functional protein comprises α-glucosidase alglucosidase alfa, α-galactosidase A (α-Gal A), beta-glucocerebrosidase, lysosomal enzyme iduronate-2-sulfatase, asfotase alfa, lysosomal acid lipase (LAL), sebelipase alfa, Iduronidase (IDUA), arylsulfatase B (ARSB), SMPD1, or Insulin.

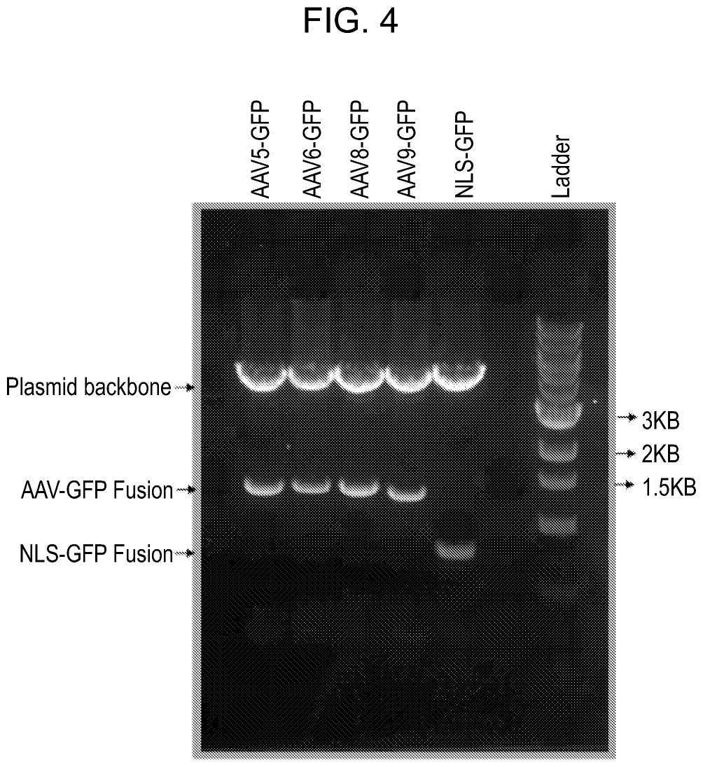

In certain embodiments, the functional protein comprises or is encoded by: CLN3 (Neuro Ceroid-Lipofuscinosis); FUCA1 (Fucosidosis); GAN (Giant Axonal Neuropathy); GALC (Globoid cell leukodystrophy); Mucolipidiosis Type IV GALC; Mucolipidiosis Type IV MCOLN1; Neuronal Ceroid Lipofusinoses PPT1; Niemann-Pick Disease SMPD1; HEXB (Sandhoff Disease); SGSH (Sanfilippo syndrome); HEXA (Tay-Sachs Disease); NEU1 (Sialidosis); SUMF1 (Multiple Sulfatase Deficiency); GAT1/SLCA1 (Childhood Epilepsy); CMT FIG. 4 (Peripheral Neuropathy); CLN5 (Neuronal Ceroid Lipofusinoses); AGA (Aspartylglycosaminuria); GDNF (Parkinsons Disease/Symptoms); GLB1 (GM1-Gangliosidosis); PMP22/MFN2 (Charcot-Marie-Tooth Type 1A); MECP2 (Retts Syndrome); LAMP2 (Dannon Disease); NAGLU (Mucopolysaccharidoses); GUSB (Sly Syndrome); SLC19A3 (Biotin basal ganglia disease); PLP1 (Pelizaeus-Merzbacher disease); TPP1/CLN2 (Neuronal Ceroid Lipofusinoses); ACY2/ASPA (Canavan Disease); MANBA (Beta-Mannosidosis); CTNS (Cystinosis); GNS (Mucopolysaccharidoses); HGSNAT (Mucopolysaccharidoses); SLC17A5 (Salla Disease); CLN6 (Jansky-Bielschowsky disease); CLN8 (Neuronal ceroid lipofuscinoses); GM2 gangliosidosis.

Another aspect of the invention provides a pharmaceutical composition comprising the engineered AAV capsid VP1 of the invention, or the AAV viral particle of the invention, and a pharmaceutically acceptable carrier or excipient.

Another aspect of the invention provides a method of treating a (genetic) disease or disorder in a subject, the method comprising introducing the AAV viral particle of the invention, or the pharmaceutical composition of the invention, into the subject.

In certain embodiments, the (genetic) disease or disorder is: (1) Pompe disease or GAA deficiency, and the GOI encodes α-glucosidase alglucosidase alfa; (2) Fabry disease or a deficiency of α-galactosidase A (α-Gal A), and the GOI encodes α-galactosidase A; (3) Gaucher disease or beta-glucocerebrosidase deficiency, and the GOI encodes beta-glucocerebrosidase; (4) Hunter syndrome or MPS-II, and the GOI encodes lysosomal enzyme iduronate-2-sulfatase; (5) hypophosphatasia (HPP), such as perinatal/infantile- and juvenile-onset HPP, and the GOI encodes asfotase alfa; (6) lysosomal acid lipase deficiency (LAL-D), and the GOI encodes lysosomal acid lipase (LAL) or sebelipase alfa; (7) Hurler syndrome/Scheie syndrome, and the GOI encodes Iduronidase (IDUA); (8) Maroteaux-Lamy syndrome, and the GOI encodes arylsulfatase B (ARSB); (9) sphingomyelinase deficiency (ASMD), and the GOI encodes SMPD1; or, (10) Type1 Diabetes, Type2 Diabetes and hyperglycemia, and the GOI encodes Insulin.

In certain embodiments, the (genetic) disease or disorder is: Neuro Ceroid-Lipofuscinosis; Fucosidosis; Giant Axonal Neuropathy; Globoid cell leukodystroph); Mucolipidiosis Type IV; Mucolipidiosis Type IV; Neuronal Ceroid Lipofusinoses; Niemann-Pick Disease; Sandhoff Disease; Sanfilippo syndrome; Tay-Sachs Disease; Sialidosis; Multiple Sulfatase Deficiency; Childhood Epilepsy; Peripheral Neuropathy; Neuronal Ceroid Lipofusinoses; Aspartylglycosaminuria; Parkinsons Disease/Symptoms; GM1-Gangliosidosis; Charcot-Marie-Tooth Type 1A; Retts Syndrome; Dannon Disease; Mucopolysaccharidoses; Sly Syndrome; Biotin basal ganglia disease; Pelizaeus-Merzbacher disease; Canavan Disease; Beta-Mannosidosis; Cystinosis; Mucopolysaccharidoses; Mucopolysaccharidoses; Salla Disease; Jansky-Bielschowsky disease; Neuronal ceroid lipofuscinoses; GM2 gangliosidosis.

Another aspect of the invention provides a method of producing the AAV viral particle of the invention, comprising introducing the polynucleotide of the invention into a packaging cell line that constitutively or inducibly expresses said polynucleotide and an AAV cap protein.

It should be understood that all embodiments of the invention, including those described only in the examples or figures, and/or only in one section of the specification, can be combined with any additional one or more embodiments of the invention, unless such combination is expressly disclaimed or is improper.

With the general aspects of the invention described herein, more specific embodiments and aspects of the inventions are described in further details in the sections that follow.

### 2. Definitions

As used herein, the term “AAV serotype” refers to the preference of a functional AAV capsid, when present in a recombinant AAV viral particle, for transducing a target cell, tissue or organ, in particular a cell, tissue, or organ of interest, and expressing a transgene (GOI) carried by the rAAV in said target cell, tissue or organ. The term AAV serotype encompasses any natural or artificial AAV capsid serotypes, including AAV capsid variants isolated from primate (human or non-human) or non-primate species and AAV capsid variants engineered by various techniques known in the art.

As used herein, the term “tropism” refers to the capacity of an AAV capsid protein present in a recombinant AAV viral particle, to transduce some particular type(s) of cell(s), tissue(s) or organ(s) (e.g., cellular or tissue tropism).

The tropism of the subject recombinant or engineered hybrid AAV capsid protein (or hybrid AAV capsid) for a particular type of cell, tissue or organ may be determined by measuring the ability of the AAV vector particles comprising the subject hybrid AAV capsid protein to transduce said particular type of cell, tissue or organ or express a transgene in said particular type of cell, tissue or organ, using standard assays that are well-known in the art.

For example, vector transduction or transgene expression can be determined by local or systemic administration of hybrid capsid serotype AAV vector particles in animal models such as mouse models that are well known in the art. Parent AAV vector serotypes comprising the donor or acceptor capsids can be used for comparison.

Vector transduction may be determined by measuring vector genome copy number per diploid genome by standard assays that are well known in the art, such as real-time PCR (RT-PCR). Transgene expression is advantageously measured using a reporter gene such as luciferase or fluorescent protein (GFP or others) by standard assays that are well known in the art, e.g., in quantitative bioluminescence or fluorescence assays in vivo or in vitro.

A “recombinant AAV” or “rAAV” is a DNase-resistant viral particle containing two elements, an AAV capsid and a vector genome containing at least a non-AAV coding sequence packaged within the AAV capsid. Unless otherwise specified, this term can be used interchangeably with the phrase “rAAV vector.”

The rAAV is a “replication-defective virus” or “viral vector,” as it lacks any functional AAV rep gene or functional AAV cap gene and cannot generate progeny. In certain embodiments, the only AAV sequences on the rAAV are the AAV's inverted terminal repeat sequences (ITRs), typically located at the extreme 5′ and 3′ ends of the vector genome in order to allow the gene and regulatory sequences located between the ITRs to be packaged within the AAV capsid.

A “vector genome” refers to the nucleic acid sequence packaged inside the rAAV capsid which forms a viral particle. Such a nucleic acid sequence contains AAV inverted terminal repeat sequences (ITRs). In certain embodiments, a vector genome contains, at a minimum, from 5′ to 3′, an AAV 5′ ITR, coding sequence(s) for a gene of interest (GOI), and an AAV 3′ ITR. ITRs from AAV2, a different source AAV than the capsid, or other than full-length ITRs may be selected. In certain embodiments, the ITRs are from the same AAV source as the AAV which provides the rep function during production or a transcomplementing AAV. In certain embodiments, other ITRs may be used. In certain embodiments, the vector genome contains regulatory sequences (such as promoters and enhancers) which direct expression of the gene products (such as those in the GOI). Suitable components of a vector genome are discussed in more detail herein.

A rAAV is composed of an AAV capsid (which comprises VP1-VP3 in a given ratio) and a vector genome (vg).

The term “identity” refers to the sequence similarity between two polypeptide molecules or between two nucleic acid molecules. When a position in both compared sequences is occupied by the same base or same amino acid residue, the respective molecules are said to be identical at that position. The percentage of identity between two sequences corresponds to the number of matching positions shared by the two sequences divided by the number of positions compared and multiplied by 100. Generally, a comparison is made when two sequences are aligned to give maximum identity. The identity may be calculated by alignment using, for example, the GCG (Genetics Computer Group, Program Manual for the GCG Package, Version 7, Madison, Wisconsin) pileup program, or any of sequence comparison algorithms such as BLAST, FASTA, CLUSTALW, MEGA and the like.

As used herein, the term “muscle” includes smooth muscle (e.g., diaphragm), cardiac muscle (i.e. heart), and skeletal muscle.

The term “muscle cells” refers to myocytes, myotubes, myoblasts, and/or satellite cells. The skeletal muscles are classified in different groups based on their anatomical position in the body. The tropism of the hybrid AAV capsid according to the invention for different muscle groups may be measured in mice Tibialis (TA), Extensor Digitorum Longus (EDL), Quadriceps (Qua), Gastrocnemius (Ga), Soleus (Sol), Triceps, Biceps and/or Diaphragm; in particular in mice Extensor Digitorum Longus (EDL), Soleus (Sol), Quadriceps (Qua), Triceps and Diaphragm or Soleus (Sol), Quadriceps (Qua), Triceps and Diaphragm muscles.

As used herein, the term “central nervous system or CNS” refers to the brain, spinal cord, retina, optic nerve, and/or olfactory nerves and epithelium. As used herein, the term CNS cells refer to any cells of the CNS including neurons and glial cells (oligodendrocytes, astrocytes, ependymal cells, microglia).

As used herein “seroprevalence” refers to the human seroprevalence, or the level of anti-AAV antibodies binding to an AAV capsid serotype present in a human population and expressed as seric antibodies or immunoglobulins. The seroprevalence of an AAV capsid is measured using a cohort of human sera and standard assays that are well known in the art and disclosed, for example, in (Meliani et al., Hum Gene Ther Methods. 26(2):45-53, 2015). The assay may be an ELISA assay. The seroprevalence of an AAV capsid serotype (or serotype) may be defined as the percentage of individuals having an ELISA titer of IgG specific for said serotype higher than 10 pg/mL. A low prevalent serotype may be defined as a serotype with less than around 30% of individuals that are seropositive, corresponding to a seroprevalence similar or lower to AAV8 capsid seroprevalence which is considered as a reference of low-seroprevalence. A high-seroprevalent AAV capsid serotype refers to a AAV capsid serotype having a seroprevalence higher than 50%. A seroprevalence equivalent to the seroprevalence of the acceptor AAV capsid refers to a seroprevalence which is around 30%. Alternatively, the seroprevalence may be defined as the dilution at which a reduction of 50% of the OD signal is observed (OD50) using a dose-response curve. The OD50 of the tested AAV capsid is compared to that of a reference AAV capsid of known seroprevalence.

As used herein, “AAV capsid from a clinically validated AAV serotype” includes capsids from natural or artificial AAV serotypes. For example, at least 13 different AAV serotypes (AAV1 to 13) have been identified in human and non-human primates and classified in various clades and clones based on phylogenetic analysis of VP1 sequences of various primate AAV isolates: AAV1 and AAV6 correspond to Clade A; AAV2 to Clade B; AAV2-AAV3 hybrid to Clade C; AAV7 to Clade D; AAV8 to Clade E; AAV9 to Clade F, whereas AAV3, AAV4 and AAV5 are disclosed as clones (Gao et ah, J. Virol., 2004, 78, 6381-6388). AAV2 variant serotypes and AAV2/13 hybrid capsids have been isolated in human liver (La Bella et al., Gut 69:737-747, 2020). Other AAV serotypes have been identified in non-primate species, such as porcine, bovine, avian and caprine. Porcine AAV includes in particular AAVpo1, po2.1, po4 to 6. Various AAV capsid variants, also named “synthetic AAV serotypes” or new AAV serotypes” have been engineered, in particular by directed gene evolution or in silico discovery such as with no limitations recombinant AAV2-derived serotypes DJ, DJ8 and PHP.B which are hybrid capsids from 8 AAV serotypes (AAV2, 4, 5, 8, 9, avian, bovine and goat) AAV-Anc80, AAV2i8, AAV-LK03 and others.

In certain embodiments, the clinically validated AAV serotype includes AAV capsid proteins from an AAV serotype that has been or planned to be used in gene therapy clinical trial, also known as “conventional AAV serotype” such as for example AAV1, AAV2, AAV2 variants (such as the quadruple-mutant capsid optimized AAV2 comprising an engineered capsid with Y44+500+730F+T491V changes, disclosed in Ling et al., Hum Gene Ther Methods., 2016), AAV3 and AAV3 variants (such as the AAV3-ST variant comprising an engineered AAV3 capsid with two amino acid changes, S663V+T492V, see Vercauteren et al., Mol. Ther. Vol. 24(6), p. 1042, 2016), -3B and AAV-3B variants, AAV4, AAV5, AAV6 and AAV6 variants (such as the AAV6 variant comprising the triply mutated AAV6 capsid Y731F/Y705F/T492V form disclosed in Rosario et al., Mol Ther Methods Clin Dev. 3, p.16026, 2016), AAV7, AAV8, AAV9, AAV 2G9, AAV 10 such as AAVcylO and AAVrh10, AAVrh32.33, AAVrh39, AAVrh43, AAVrh74, AAV-DJ, AAVAnc80, AAV-LK03, AAV.PHP such as AAV-PHP.B, AAV-PHP.EB, AAV2i8, clade F AAVHSC such as AAVHSC7, AAVHSC15 and AAVHSC17, AAV9.rh74 and AAV9.rh74-P1 (WO 2019/193119, incorporated by reference), porcine AAV such as AAVpo1, AAVpo2.1, AAVpo4 and AAVpo6, and tyrosine, lysine and serine capsid mutants of AAV serotypes.

In particular embodiments, the clinically validated AAV serotype includes an AAV serotype selected from the group consisting of: AAV4, AAV5, AAV7, AAV8, AAV9, AAVrh10, AAVrh32.33, AAVrh39, AAVrh43, AAVrh74, AAV9.rh74, AAV9.rh74-P1, AAV-DJ, AAVAnc80, AAV2i8, AAV-LK03, and AAV.PHP. AAV4 capsid (GenBank accession number NC_001829.1); AAV5 capsid (GenBank accession number NC_006152.1); AAV7 capsid (GenBank accession number NC_006260.1); AAV9 capsid (GenBank accession number AY530579.1); AAVrh10 capsid (GenBank accession number AY243015.1); AAV-LK03, AAVrh74, AAV9.rh74, AAV9.rh74-Pl.

In particular embodiments, the clinically validated AAV serotype is a newly-isolated natural AAV variant serotype such as, for example, AAV2/13 hybrid serotype, in particular isolated from human tissue such as liver tissue. In certain embodiments, the clinically validated AAV serotype is from an AAV serotype used in gene therapy, such as AAV13 (GenBank accession number EU285562.1; or GenBank accession number ABZ10812.1).

In some embodiments, the hybrid AAV capsid protein of the invention has tropism for muscles and/or the central nervous system. In some embodiments, the hybrid AAV capsid protein has tropism for kidney. In some embodiments, the hybrid AAV capsid protein has tropism for heart and/or skeletal muscles. In certain embodiments, the hybrid AAV capsid protein has increased tropism for different skeletal muscle groups. In certain embodiments, the hybrid AAV capsid protein has tropism for at least two skeletal muscle groups in mice selected from the group consisting of: Extensor Digitorum Longus (EDL), Soleus (Sol), Quadriceps (Qua), Triceps and Diaphragm or Soleus (Sol), Quadriceps (Qua), Triceps and Diaphragm. In some particular embodiments, the hybrid AAV capsid protein has a decreased tropism for an off-target tissue, advantageously the liver.

### 3. AAV Vector Genomes, Viral Particles, Polynucleotides, and Host Cells/Packaging Cells

One aspect of the invention provides a polynucleotide encoding the engineered or hybrid capsid VP polypeptides of the invention.

In certain embodiments, the polynucleotide may be DNA, RNA, or a synthetic or semi-synthetic nucleic acid.

In some embodiments, the polynucleotide comprises or consists of the sequence of SEQ ID NO: 1, and a sequence having at least 80%, 85%, 90%, 95%, 97%, 98% or 99% identity with SEQ ID NO: 1. The polynucleotide is a functional polynucleotide sequence, in that it codes for the hybrid AAV capsid protein of the invention.

In some embodiments, the polynucleotide further encodes an AAV Replicase (Rep) protein in expressible form, preferably Rep from AAV2.

In a related aspect, the invention provides a vector comprising the polynucleotide of the invention. That is, in certain embodiments, the polynucleotide of the invention is inserted into a recombinant vector, which includes, in a non-limiting manner, linear or circular DNA or RNA molecules consisting of chromosomal, non-chromosomal, synthetic or semi-synthetic nucleic acids, such as in particular viral vectors, plasmid or RNA vectors.

Numerous vectors into which a nucleic acid molecule of interest can be inserted in order to introduce it into and maintain it in a eukaryotic host cell are known in the art, with the choice of an appropriate vector partly depends on the use envisioned for this vector (for example, replication of the sequence of interest, expression of this sequence, maintaining of this sequence in extrachromosomal form, or else integration into the chromosomal material of the host), and also on the nature of the host cell.

In some embodiments, the vector is a plasmid. In certain embodiments, the plasmid is a DNA plasmid comprising coding sequence for the hybrid capsid of the invention (such as an repcap plasmid encoding an AAV2 Rep and the AAV9-8 hybrid capsid as illustrated in Example 2). The DNA plasmids can be transferred to cells permissible for infection with a helper virus of AAV (e.g., adenovirus, E1-deleted adenovirus or herpes virus) for assembly of the rAAV genome into infectious viral particles comprising the subject hybrid capsid, such as AAV9-8.

In certain embodiments, the vector is an expression vector comprising appropriate means for expression of the hybrid AAV capsid protein, and optionally also an AAV Rep protein (such as an AAV2 Rep). In certain embodiments, each coding sequence (hybrid AAV Cap and AAV Rep) is inserted in a separate expression cassette, either in the same vector or separately. Each expression cassette comprises the coding sequence (open reading frame or ORF) functionally linked to the regulatory sequences which allow the expression of the corresponding protein in AAV producer cells, such as in particular promoter, promoter/enhancer, intron, initiation codon (ATG), stop codon, transcription termination signal. Alternatively, in other embodiments, the hybrid AAV Cap and the AAV Rep proteins may be expressed from a unique expression cassette using an Internal Ribosome Entry Site (IRES) inserted between the two coding sequences or a viral 2A peptide. In certain embodiments, the codon sequences encoding the hybrid AAV Cap, and AAV Rep (if present), are optimized for expression in AAV producer cells, in particular human producer cells such as HEK293T cells.

Another related aspect of the invention provides the engineered/hybrid VP polypeptides of the invention.

Another related aspect of the invention provides a recombinant AAV viral particle comprising the engineered/hybrid VP polypeptides of the invention, encapsidating an AAV vector genome (vg) comprising a gene-of-interest (GOI) flanked by an AAV ITR sequences (such as the 5′ and 3′ ITR sequences of AAV2).

In certain embodiments, the AAV viral particle is a recombinant AAV (rAAV) vector particle, also named hybrid capsid serotype rAAV vector particle or hybrid serotype rAAV vector particle. In other words, recombinant AAV (i.e., infectious encapsidated rAAV particles) of the invention comprise a rAAV vector genome comprising a GOI, encapsidated in a hybrid capsid of the invention.

In certain embodiments, to promote skeletal muscle specific expression, AAV-1, AAV-5, AAV-6, AAV-rh74, AAV-8 or AAV-9, for example, may be used as one of the parental capsids for generating the hybrid capsid of the invention. The Examples illustrate the use of AAV9 as such a parental capsid. Substantially similar approaches can be used for the other parental capsid having natural tropism for muscle and/or neuronal cells.

In certain embodiments, the AAV viral particle has a serotype with muscle tropism, either exclusively, or preferentially.

The AAV vector particle is suitable for gene therapy directed to a target tissue or cells in the individual, in particular muscle, and/or CNS cells or tissue or other cells or tissues. The rAAV vector particle is packaging a gene of interest (GOI). The genome of the rAAV vector may either be a single-stranded or self-complementary double-stranded genome (McCarty et al, Gene Therapy 10(26):2112-2118, 2003).

In certain embodiments, the vector genome is a self-complementary vector. Self-complementary vectors are generated by deleting the terminal resolution site (trs) from one of the AAV terminal repeats. These modified vectors, whose replicating genome is half the length of the wild-type AAV genome have the tendency to package DNA dimers.

The AAV genome is flanked by ITRs. In particular embodiments, the AAV vector is a pseudotyped vector, i.e., its genome and capsid are derived from AAVs of different serotypes. In some embodiments, the genome of the pseudotyped vector is derived from AAV2.

The rAAV vector particle may be obtained using the method of producing recombinant AAV vector particles of the invention (see below). More specifically, production of pseudotyped rAAV is disclosed in, for example, WO 01/83692. Other types of rAAV variants, for example rAAV with capsid mutations such as the hybrid capsids of the invention, can also be produced similarly. See, for example, Marsic et al., Molecular Therapy, 22(11): 1900-09 (2014). The nucleotide sequences of the genomes of various AAV serotypes are known in the art.

Another related aspect of the invention provides a cells, such as a host cell or a packaging cell for AAV viral production, comprising the polynucleltide, hybrid capsid polypeptide, vector, or viral particle of the invention.

In certain embodiments, the cell is an isolated cell from an individual, which is stably transduced with a rAAV vector particle of the invention. In certain embodiments, the individual is a patient to be treated. In some embodiments, the cell is a muscle and/or CNS cell according to the present disclosure, or a progenitor of said cell, or a pluripotent stem cell such as induced pluripotent stem cell (iPS cell), embryonic stem cells, fetal stem cell and adult stem cell.

In certain embodiments, the cell is a packaging cell for AAV production.

A method of generating a packaging cell is to create a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., Proc. Natl. Acad. S6. USA 79:2077-2081, 1982), addition of synthetic linkers containing restriction endonuclease cleavage sites (Laughlin et al., Gene, 23:65-73, 1983) or by direct, blunt-end ligation (Senapathy & Carter, J. Biol. Chem, 259:4661-4666, 1983). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

The invention thus provides packaging cells that produce infectious rAAV. In one embodiment packaging cells may be stably transformed cancer cells such as HeLa cells, 293 cells and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

In certain embodiments, the cell is stably transformed with a recombinant vector for expression of the hybrid AAV capsid protein, and optionally also an AAV Rep protein. The cell stably expresses the hybrid AAV capsid and optionally the AAV Rep proteins may be referred to as a producer cell line. The producer cell is optionally a human cell, such as HEK293T cells.

The vector, such as a recombinant plasmid, and the producer cell line are useful for producing hybrid AAV vectors comprising the hybrid AAV capsid protein of the invention, using standard AAV production methods that are well-known in the art (see Aponte-Ubillus el al., Applied Microbiology and Biotechnology 102:1045-1054, 2018).

Techniques to produce rAAV particles, in which an AAV vector genome to be packaged, rep and cap genes, and helper virus functions are provided to a cell are standard in the art, and is commonly known as the so-called the “triple transfection” method, even though the method of introduction may not necessarily be the traditional means for plasmid transfection.

Production of rAAV generally requires that the following components be present within a single cell (denoted herein as a packaging cell): a rAAV genome, AAV rep and cap genes separate from (i.e., not in) the rAAV genome, and helper virus functions.

Briefly, following co-transfection of the producer cell line stably expressing the hybrid AAV capsid and AAV Rep proteins with plasmid containing recombinant AAV vector genome comprising the gene of interest inserted in an expression cassette, flanked by AAV ITRs, in the presence of sufficient helper function to permit packaging of the rAAV vector genome into AAV capsid particle, the cells are incubated for a time sufficient to allow the production of AAV vector particles, the cells are then harvested, lysed, and AAV vector particles are purified by standard purification methods such as column chromatography (e.g., affinity chromatography) or Iodixanol or Cesium Chloride density gradient ultracentrifugation.

Methods for purifying rAAV vectors from helper virus are known in the art and include methods disclosed in, for example, Clark et al., Hum. Gene Ther., 10(6): 1031-1039 (1999); Schenpp and Clark, Methods Mol. Med., 69427-443 (2002); U.S. Pat. No. 6,566,118 and WO 98/09657.

Additional general principles of rAAV production are reviewed in, for example, Carter, 1992, Current Opinions in Biotechnology, 1533-539; and Muzyczka, 1992, Curr. Topics in Microbial, and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595. The foregoing documents are hereby incorporated by reference in their entirety herein, with particular emphasis on those sections of the documents relating to rAAV production.

### 4. Composition and Pharmaceutical Composition

Another aspect of the invention provides compositions comprising the engineered AAV capsid (e.g., VP1, VP2, and VP3) of the invention, the rAAV viral particles of the invention, and a pharmaceutically acceptable carrier or excipient. Compositions described herein comprise rAAV in a pharmaceutically acceptable carrier. The compositions may also comprise other ingredients such as diluents and adjuvants.

A “pharmaceutically acceptable carrier” refers to a vehicle that does not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In certain embodiments, the pharmaceutical composition contains vehicles or carriers, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The rAAV vector particle, cell and derived pharmaceutical composition of the invention may be used for treating diseases by gene therapy, in particular targeted gene therapy directed to muscle and/or CNS cells or tissue. The cell and derived pharmaceutical composition of the invention may be used for treating diseases by cell therapy, in particular cell therapy directed to muscle and/or CNS cell or other target cells of interest.

Acceptable carriers, diluents and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycol (PEG).

Titers of rAAV to be administered in methods of the invention will vary depending, for example, on the particular rAAV, the mode of administration, the treatment goal, the individual, and the cell type(s) being targeted, and may be determined by methods standard in the art.

Exemplary titers of rAAV may range from about 1×10⁶, about 1×10⁷, about 1×10⁸, about 1×10⁹, about 1×10¹⁰, about 1×10¹¹, about 1×10¹², about 1×10¹³ to about 1×10¹⁴ or more DNase resistant particles (DRP) per ml.

Dosages may also be expressed in units of viral genomes (vg). The titers of rAAV may be determined by the supercoiled plasmid quantitation standard or the linearized plasmid quantitation standard.

In certain embodiment, the disclosure provides methods of measuring the titer of an AAV vector, comprising tittering the AAV vector with PCR with a first primer and a second primer. In another embodiment, methods of measuring the titer of an AAV vector, comprising tittering the AAV vector with a probe.

Methods of transducing a target cell with rAAV, in vivo or in vitro, are contemplated by the invention. The in vivo methods comprise the step of administering an effective dose, or effective multiple doses, of a composition comprising a rAAV of the invention to an animal (including a human being) in need thereof. If the dose is administered prior to development of a disorder/disease, the administration is prophylactic. If the dose is administered after the development of a disorder/disease, the administration is therapeutic. In embodiments of the invention, an effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival. An example of a disease contemplated for prevention or treatment with methods of the invention is a muscular dystrophy disease (such as DMD/BMD), a neuronal disease, or a disease that can be treated by expression of a secreted protein by muscle cells.

Pharmaceutical compositions of the invention can be prepared as injectable formulations or as topical formulations to be delivered to the muscles by transdermal transport. Numerous formulations for both intramuscular injection and transdermal transport have been previously developed and can be used in the practice of the invention.

In general, the pharmaceutical forms suitable for injectable use include sterile aqueous solutions or suspensions. The solution or suspension may comprise additives which are compatible with viral vectors and do not prevent viral vector particle entry into target cells. In all cases, the form must be sterile and must be fluid to the extent that easy syringe ability exists. It must be stable under the conditions of manufacture and storage and must be preserved against the contaminating action of microorganisms, such as bacteria and fungi. An example of an appropriate solution is a buffer, such as phosphate buffered saline (PBS) or Ringer lactate.

The rAAV can be used with any pharmaceutically acceptable carrier for ease of administration and handling. Thus, in another aspect, the application is directed to a formulation that comprises an rAAV that comprises a subject hybrid AAV capsid with muscle and/or neuronal tissue tropism, such as AAV9-8, a buffer agent, an ionic strength agent, and a surfactant.

In one embodiment, the rAAV is at a concentration of 1.0×10¹² vg/mL to about 1.0×10¹⁶ vg/mL, or about 1.0×10¹² vg/mL to about 5.0×10¹⁴ vg/mL.

In another embodiment, the rAAV is at a concentration of about 5.0×10¹² vg/mL to about 1.0×10¹⁴ vg/mL based on a supercoiled plasmid as the quantitation standard.

In another embodiment, the rAAV is at a concentration of about 5.0×10¹² vg/mL to about 1.0×10¹⁴ vg/mL based on a linearized plasmid as the quantitation standard.

In another embodiment, the rAAV is at a concentration of about 2.0×10¹³ vg/mL based on a supercoiled plasmid as the quantitation standard.

In one embodiment, the concentration of rAAV in the composition or formulation is from 1.0×10¹³ vg/mL to 2.0×10¹⁴ vg/mL based on a supercoiled plasmid as the quantitation standard.

In another embodiment, the concentration is 2.0×10¹³ vg/mL, 4.0×10¹³ vg/mL, or 5.0×10¹³ vg/mL based on a supercoiled plasmid as the quantitation standard.

In one embodiment, the buffer agent comprises one or more of tris, tricine, Bis-tricine, HEPES, MOPS, TES, TAPS, PIPES, and CAPS.

In another embodiment, the buffer agent comprises tris with pH 8.0 at concentration of about 5 mM to about 40 M.

In one embodiment, the buffer agent comprises tris with pH 8.0 at about 20 mM.

In one embodiment, the ionic strength agent comprises one of more of potassium chloride (KCl), potassium acetate, potassium sulfate, ammonium sulfate, ammonium chloride (NH₄Cl), ammonium acetate, magnesium chloride (MgCl₂), magnesium acetate, magnesium sulfate, manganese chloride (MnCl₂), manganese acetate, manganese sulfate, sodium chloride (NaCl), sodium acetate, lithium chloride (LiCl), and lithium acetate.

In one embodiment, the ionic strength agent comprises MgCl₂ at a concentration of about 0.2 mM to about 4 mM. In another embodiment, the ionic strength agent comprises NaCl at a concentration of about 50 mM to about 500 mM. In another embodiment, the ionic strength agent comprises MgCl₂ at a concentration of about 0.2 mM to about 4 mM and NaCl at a concentration of about 50 mM to about 500 mM. In another embodiment, the ionic strength agent comprises MgCl₂ at a concentration of about 1 mM and NaCl at a concentration of about 200 mM.

In one embodiment, the surfactant comprises one or more of a sulfonate, a sulfate, a phosphonate, a phosphate, a Poloxamer, and a cationic surfactant.

In one embodiment, the Poloxamer comprises one or more of Poloxamer 124, Poloxamer 181, Poloxamer 184, Poloxamer 188, Poloxamer 237, Poloxamer 331, Poloxamer 338, and Poloxamer 407.

In one embodiment, the surfactant comprises the Poloxamer at a concentration of about 0.00001% to about 1%.

In another embodiment, the surfactant comprises Poloxamer 188 at a concentration of about 0.001%.

For purposes of intramuscular injection, solutions in an adjuvant such as sesame or peanut oil or in aqueous propylene glycol can be employed, as well as sterile aqueous solutions. Such aqueous solutions can be buffered, if desired, and the liquid diluent first rendered isotonic with saline or glucose. Solutions of rAAV as a free acid (DNA contains acidic phosphate groups) or a pharmacologically acceptable salt can be prepared in water suitably mixed with a surfactant such as hydroxpropylcellulose. A dispersion of rAAV can also be prepared in glycerol, liquid polyethylene glycols and mixtures thereof and in oils.

Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, phenol, chlorobutanol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

### 5. Administration

Administration of an effective dose of the compositions may be by routes standard in the art including, but not limited to, intramuscular, parenteral, intravenous, oral, buccal, nasal, pulmonary, intracranial, intraosseous, intraocular, rectal, or vaginal. Route(s) of administration and serotype(s) of AAV components of the rAAV (in particular, the AAV ITRs and capsid protein) of the invention may be chosen and/or matched by those skilled in the art taking into account the infection and/or disease state being treated and the target cells/tissue(s) that are to express the E-insulin.

The invention provides for local administration and systemic administration of an effective dose of rAAV and compositions of the invention. For example, systemic administration is administration into the circulatory system so that the entire body is affected. Systemic administration includes enteral administration such as absorption through the gastrointestinal tract and parental administration through injection, infusion or implantation.

For example, administration of rAAV of the present invention may be accomplished by using any physical method that will transport the rAAV recombinant vector into the target tissue of an animal. Administration according to the invention includes, but is not limited to, injection into muscle, the bloodstream and/or directly into the liver. Simply resuspending a rAAV in phosphate buffered saline has been demonstrated to be sufficient to provide a vehicle useful for muscle tissue expression, and there are no known restrictions on the carriers or other components that can be co-administered with the rAAV (although compositions that degrade DNA should be avoided in the normal manner with rAAV). Capsid proteins of a rAAV may be modified so that the rAAV is targeted to a particular target tissue of interest such as muscle. See, for example, WO 02/053703, the disclosure of which is incorporated by reference herein.

Transduction with rAAV may also be carried out in vitro. In one embodiment, desired target muscle cells are removed from the subject, transduced with rAAV and reintroduced into the subject. Alternatively, syngeneic or xenogeneic muscle cells can be used where those cells will not generate an inappropriate immune response in the subject.

Suitable methods for the transduction and reintroduction of transduced cells into a subject are known in the art. In one embodiment, cells can be transduced in vitro by combining rAAV with muscle cells, e.g., in appropriate media, and screening for those cells harboring the DNA of interest using conventional techniques such as Southern blots and/or PCR, or by using selectable markers. Transduced cells can then be formulated into pharmaceutical compositions, and the composition introduced into the subject by various techniques, such as by intramuscular, intravenous, subcutaneous and intraperitoneal injection, or by injection into smooth and cardiac muscle, using e.g., a catheter.

Transduction of cells with rAAV of the invention results in sustained expression of the GOI.

The present invention thus provides methods of administering/delivering rAAV which express a given GOI to a mammalian subject, preferably a human being. These methods include transducing tissues (including, but not limited to, tissues such as muscle, organs such as liver and brain, and glands such as salivary glands) with one or more rAAV of the present invention. Transduction may be carried out with gene cassettes comprising tissue specific control elements.

For example, one embodiment of the invention provides methods of transducing muscle cells and muscle tissues directed by muscle specific control elements, including, but not limited to, those derived from the actin and myosin gene families, such as from the myoD gene family [See Weintraub et al., Science, 251: 761-766 (1991)], the myocyte-specific enhancer binding factor MEF-2 [Cseijesi and Olson, Mol Cell Biol 11: 4854-4862 (1991)], control elements derived from the human skeletal actin gene [Muscat et al., Mol Cell Biol, 7: 4089-4099 (1987)], the cardiac actin gene, muscle creatine kinase sequence elements [See Johnson et al., Mol Cell Biol, 9:3393-3399 (1989)] and the murine creatine kinase enhancer (mCK) element, control elements derived from the skeletal fast-twitch troponin C gene, the slow-twitch cardiac troponin C gene and the slow-twitch troponin I gene: hypoxia-inducible nuclear factors (Semenza et al., Proc Natl Acad Sci USA, 88: 5680-5684 (1991)), steroid-inducible elements and promoters including the glucocorticoid response element (GRE) (See Mader and White, Proc. Natl. Acad. Sci. USA 90: 5603-5607 (1993)), and other control elements.

Muscle tissue is an attractive target for in vivo DNA delivery, because it is not a vital organ and is easy to access. The invention contemplates sustained expression of a GOI mRNAs from transduced myofibers.

By “muscle cell” or “muscle tissue” is meant a cell or group of cells derived from muscle of any kind (for example, skeletal muscle and smooth muscle, e.g. from the digestive tract, urinary bladder, blood vessels or cardiac tissue). Such muscle cells may be differentiated or undifferentiated, such as myoblasts, myocytes, myotubes, cardiomyocytes and cardiomyoblasts.

The term “transduction” is used to refer to the administration/delivery of a polynucleotide of interest (e.g., a polynucleotide sequence encoding the GOI) to a recipient cell either in vivo or in vitro, via a replication-deficient rAAV described resulting in expression of the GOI by the recipient cell.

Thus, also described herein are methods of administering an effective dose (or doses, administered essentially simultaneously or doses given at intervals) of rAAV that encode GOI to a mammalian subject in need thereof.

### 6. Gene-of-Interest

“Gene of interest” or “GOI,” as used herein, broadly refers to any gene useful for a particular application, such as, without limitation, diagnosis, reporting, modifying, therapy and genome editing.

For example, the gene of interest may be a therapeutic gene, a reporter gene or a genome-editing enzyme. In certain embodiments, the gene-of-interest is a functional protein (such as a therapeutic protein), a functional nucleic acid, and/or an antibody or a portion thereof (such as a heavy chain and/or a light chain of the antibody).

“Gene of interest for therapy,” “gene of therapeutic interest,” or “heterologous gene of interest,” includes a therapeutic gene or a gene encoding a therapeutic protein, peptide or RNA.

The gene of interest is any nucleic acid sequence capable of modifying a target gene or target cellular pathway, in cells of target organs, in particular muscle and/or CNS, or other target organs of interest. For example, the gene may modify the expression, sequence or regulation of the target gene or cellular pathway. In some embodiments, the gene of interest is a functional version of a gene or a fragment thereof. The functional version of said gene includes the wild-type gene, a variant gene such as variants belonging to the same family and others, or a truncated version, which preserves the functionality of the encoded protein at least partially. A functional version of a gene is useful for replacement or additive gene therapy to replace a gene, which is deficient or non-functional in a patient. In other embodiments, the gene of interest is a gene which inactivates a dominant allele causing an autosomal dominant genetic disease. A fragment of a gene is useful as recombination template for use in combination with a genome editing enzyme.

Alternatively, the gene of interest may encode a functional protein of interest for a particular application, (for example an antibody or antibody fragment, a genome-editing enzyme) or a RNA. In some embodiments, the protein is a therapeutic protein including a therapeutic antibody or antibody fragment, or a genome-editing enzyme. In some embodiments, the RNA is a therapeutic RNA.

In certain embodiments, the functional protein comprises a CRISPR/Cas effector enzyme (such as Cas9, Cas12, or Cas13), and/or wherein the functional nucleic acid is a single guide RNA (sgRNA), siRNA, miRNA, shRNA, antisense RNA, ribozyme, or aptamer.

In certain embodiments, the functional protein comprises α-glucosidase alglucosidase alfa, α-galactosidase A (α-Gal A), beta-glucocerebrosidase, lysosomal enzyme iduronate-2-sulfatase, asfotase alfa, lysosomal acid lipase (LAL), sebelipase alfa, Iduronidase (IDUA), arylsulfatase B (ARSB), SMPD1, or Insulin.

In certain embodiments, the functional protein comprises or is encoded by: CLN3 (Neuro Ceroid-Lipofuscinosis); FUCA1 (Fucosidosis); GAN (Giant Axonal Neuropathy); GALC (Globoid cell leukodystrophy); Mucolipidiosis Type IV GALC; Mucolipidiosis Type IV MCOLN1; Neuronal Ceroid Lipofusinoses PPT1; Niemann-Pick Disease SMPD1; HEXB (Sandhoff Disease); SGSH (Sanfilippo syndrome); HEXA (Tay-Sachs Disease); NEU1 (Sialidosis); SUMF1 (Multiple Sulfatase Deficiency); GAT1/SLCA1 (Childhood Epilepsy); CMT FIG. 4 (Peripheral Neuropathy); CLN5 (Neuronal Ceroid Lipofusinoses); AGA (Aspartylglycosaminuria); GDNF (Parkinsons Disease/Symptoms); GLB1 (GM1-Gangliosidosis); PMP22/MFN2 (Charcot-Marie-Tooth Type 1A); MECP2 (Retts Syndrome); LAMP2 (Dannon Disease); NAGLU (Mucopolysaccharidoses); GUSB (Sly Syndrome); SLC19A3 (Biotin basal ganglia disease); PLP1 (Pelizaeus-Merzbacher disease); TPP1/CLN2 (Neuronal Ceroid Lipofusinoses); ACY2/ASPA (Canavan Disease); MANBA (Beta-Mannosidosis); CTNS (Cystinosis); GNS (Mucopolysaccharidoses); HGSNAT (Mucopolysaccharidoses); SLC17A5 (Salla Disease); CLN6 (Jansky-Bielschowsky disease); CLN8 (Neuronal ceroid lipofuscinoses); GM2 gangliosidosis.

In some embodiments, the sequence of the gene of interest is optimized for expression in the treated individual, preferably a human individual. Sequence optimization may include a number of changes in a nucleic acid sequence, including codon optimization, increase of GC content, decrease of the number of CpG islands, decrease of the number of alternative open reading frames (ARFs) and/or decrease of the number of splice donor and splice acceptor sites.

The gene of interest is a functional gene able to produce the encoded protein, peptide or RNA in the target cells of the disease, in particular muscle cells and/or cells of the CNS or other target cells of interest. In some embodiments, the gene of interest is a human gene. The AAV viral vector comprises the gene of interest in a form expressible in cells of target organs, in particular muscle cells, including cardiac and skeletal muscle cells muscles, and/or cells of the CNS or other target cell of interest.

In certain embodiments, the gene of interest is operably linked to appropriate regulatory sequences for expression of a transgene in the individual's target cells, tissue(s) or organ(s). Such sequences which are well-known in the art include in particular a promoter, and further regulatory sequences capable of further controlling the expression of a transgene, such as without limitation, enhancer, terminator, intron, silencer, in particular tissue-specific silencer, and microRNA.

In certain embodiments, the gene of interest is operably linked to a ubiquitous, tissue-specific, constitutive, synthetic, or inducible promoter, which is functional in cells of target organs, in particular muscle and/or CNS.

The gene of interest may be inserted in an expression cassette further comprising additional regulatory sequences.

Examples of ubiquitous promoters include the CAG promoter, phosphogly cerate kinase 1 (PGK) promoter, the cytomegalovirus enhancer/promoter (CMV), the SV40 early promoter, the retroviral Rous sarcoma virus (RSV) LTR promoter, the dihydrofolate reductase promoter, the b-actin promoter, and the EF1 promoter.

In certain embodiments, the promoter is a constitutive promoter. In certain embodiments, the constitutive promoter is: CAG, CB, CB6, respiratory syncytial virus (RSV) promoter, cytomegalovirus (CMV) promoter, or elongation factor 1a (EF1a) promoter.

In certain embodiments, the promoter is a synthetic promoter. Synthetic promoter is a groundbreaking approach in promoter design. This strategy enables one to engineer promoters with defined properties, such as size and the expression profile of the transgene. The development of synthetic promoters relies on computational algorithms, which are used to identify regulatory sequences and TFBSs within the genome, as well as to predict the promoter regions. For example, the binding sites for myogenic TFs are usually shorter than 10 bp, which allows one to create a library of constructs with different combinations of muscle-specific TFBSs.

In certain embodiments, the synthetic promoter is the SPc5-12 promoter (Li et al., Nat. Biotechnol. 17(3):241-245, 1999). The SPc5-12 promoter consists of a combination of four muscle-specific TFBSs (TEF1, SRE, MEF1, and MEF2) and the core promoter (a fragment of the promoter of the chicken skeletal muscle α-actin gene). Its activity in muscle fibers is reportedly six-fold higher than that of the CMV promoter. Further, in vivo experiments confirmed that the SPc5-12 promoter is inactive in undifferentiated myoblasts and in various nonmuscle cell lines.

In certain embodiments, the synthetic promoter is the SP-301 promoter (Liu et al., Plasmid. 106:102441, 2019). The SP-301 promoter is a combination of muscle-specific TFBSs, viral elements, and conserved cis-regulatory elements ligated in forward and reverse orientation. The SP-301 promoter is 6.6 times more active than the CMV promoter 2 days after intramuscular delivery of the construct in mice and remained active for at least a month. The tissue specificity of the SP-301 promoter was confirmed in transgenic mice.

In certain embodiments, the synthetic promoter is the MH promoter. The MH promoter consists of the human desmin gene enhancer linked to the enhancer, the core promoter, and the first intron of the mouse Ckm gene. The MH promoter ensured the highest expression level in the muscle cell culture, being superior to the desmin and CMV promoters. AAV2/9 carrying a reporter gene delivered intravenously in mice under the control of the MH promoter has higher reporter activity in the cardiac and skeletal muscles than that of the desmin and CMV promoters. Further, the MH promoter does not induce transgene expression in the liver.

In certain embodiments, the synthetic promoter is the Sk-CRM4/Des promoter, which is the regulatory module Sk-CRM4 ligated to the desmin promoter and the MVM intron. Six weeks after systemic delivery using AAV9, the Sk-CRM4 chimeric promoter enhanced the activity of the desmin promoter by 200-400 times in different skeletal muscles, the diaphragm, and the heart, while remaining inactive in non-target tissues. Moreover, the SkCRM4/Des promoter attained a 25-173 times higher expression in different muscles as compared to the CMV promoter and also outperformed the Sk-CRM4/SPc5-12 and SPc5-12 promoters. Therefore, the computationally designed Sk-CRM4/Des chimeric promoter demonstrated improved muscle-specific performance as compared to the other promoters commonly used for muscle gene therapy.

In certain embodiments, the promoter is a muscle-specific promoter.

The term “muscle specific promoter or control element” refers to a nucleotide sequence that regulates expression of a coding sequence that is specific for expression in muscle tissue. These control elements include enhancers and/or promoters. Exemplary muscle specific control elements include a MCKH7 promoter, an MCK promoter, and a MCK enhancer.

In certain embodiments, muscle-specific promoters include, without limitation, the desmin (Des) promoter, muscle creatine kinase (MCK) promoter, CK6 promoter, alpha-myosin heavy chain (alpha-MHC) promoter, myosin light chain 2 (MLC-2) promoter, cardiac troponin C (cTnC) promoter, synthetic muscle-specific SpC5-12 promoter, the human skeletal actin (HSA) promoter.

In certain embodiments, the muscle-specific promoter is MHCK7, CK8e, tMCK, (human) skeletal α-actin (HSA) promoter (including full length or shortened HAS promoter, and chimeric HSA/CMV promoter consisting of a fragment of the HSA promoter and the CMV promoter), muscle creatine kinase (MCK/CKM) promoter or derivative thereof (including CK6, MHCK7, dMCK, tMCK, CK8 and CK8e), desmin gene promoter, human α-myosin heavy chain gene (aMHC) promoter, myosin light-chain promoter (MLC2v), cardiac troponin T promoter (cTnT), a chimeric promoter comprising the CMV-IE enhancer ligated to the 1.5-kbp fragment of the rat promoter MLC, and the AUSEx3 promoter developed from the human troponin I (TNNI1) gene. See Skopenkova et al., Muscle-Specific Promoters for Gene Therapy, Acta Naturae. 13(1): 47-58, 2021 (incorporated herein by reference).

Other muscle-specific promoters or control elements include human skeletal actin gene element, cardiac actin gene element, myocyte-specific enhancer binding factor MEF, muscle creatine kinase (MCK), tMCK (truncated MCK), myosin heavy chain (MHC), MHCK7 (a hybrid version of MHC and MCK), C5-12 (synthetic promoter), murine creatine kinase enhancer element, skeletal fast-twitch troponin C gene element, slow-twitch cardiac troponin C gene element, slow-twitch troponin I gene element, hypoxia-inducible nuclear factors, steroid-inducible element or glucocorticoid response element (GRE).

In certain embodiments, the promoter is a CNS-specific promoter. Promoters for CNS expression include promoters driving ubiquitous expression and promoters driving expression into neurons. Representative promoters driving ubiquitous expression include, without limitation: CAG promoter (includes the cytomegalovirus enhancer/chicken beta actin promoter, the first exon and the first intron of the chicken beta-actin gene and the splice acceptor of the rabbit beta-globin gene); PGK (phosphogly cerate kinase 1) promoter; 3-actin promoter; EF1a promoter; CMV promoter.

Representative promoters driving expression into neurons include, without limitation, the promoter of the Calcitonin Gene-Related Peptide (CGRP), a known motor neuron-derived factor. Other neuron-selective promoters include the promoters of Choline Acetyl Transferase (ChAT), Neuron Specific Enolase (NSE), Synapsin, Hb9 and ubiquitous promoters including Neuron-Restrictive Silencer Elements (NRSE).

Representative promoters driving selective expression in glial cells include the promoter of the Glial Fibrillary Acidic Protein gene (GFAP).

In certain embodiments, the GOI further encodes a 5′-UTR region, a 3′-UTR region, and/or a polyA signal operably linked to the polynucleotide of the invention.

In certain embodiments, the 5′-UTR region comprises an intron. Expression of the GOI can be enhanced due to the presence of an intron in the vector. The intron is typically positioned between the promoter and the coding region. While not wishing to be bound by any particular theory, the presence of the intron is believed to increase RNA stability in the nucleus due to the incorporation of mRNA into the spliceosome and promotes efficient export of spliced mRNA from the nucleus to the cytoplasm.

In certain embodiments, the introns contains regulatory sequences that affect tissue specificity and the expression level of the target gene. In certain embodiments, the intron is from the Ckm gene. In certain embodiments, the intron is the MVM intron.

In certain embodiments, the 3′-UTR comprises a (600-bp) post-transcriptional regulatory element of the woodchuck hepatitis virus (WPRE) which can lead to an enhancement of transgene expression in muscles. WPRE is believed to promote mRNA export from the nucleus and prevents post-translational gene silencing.

In certain embodiments, the GOI is further linked to an miRNA detargeting sequence in the AAV vector of the invention, which miRNA suppresses expression of the GOI in a non-target tissue, such as liver or neuronal tissues. For this purpose, the binding sites for certain microRNA that are present only in the non-target organs are added to the 3′-UTR of the expression cassette (e.g., miRNA detargeting sites). If transgenic mRNA is expressed in a non-target organ, microRNA binds to such complementary detargeting sites on the transgene and initiates its degradation.

In certain embodiments, the GOI encodes a functional nucleic acid, such as RNA. In certain embodiments, the GOI encodes a therapeutic RNA, such as an interfering RNA like a shRNA or a microRNA, a guide RNA (gRNA) for use in combination with a Cas enzyme or similar enzyme, or an antisense RNA capable of exon skipping such as a modified small nuclear RNA (snRNA); and a gene encoding a genome-editing enzyme, such as an engineered nuclease like a meganuclease, zinc finger nuclease (ZFN), transcription activator-like effector-based nuclease (TALENs), Cas enzyme or similar enzymes; or a combination thereof, or a fragment of a functional version of a gene for use as recombination template.

In certain embodiments, the RNA is complementary to a target DNA or RNA sequence, or binds to a target protein. For example, in some embodiments, the RNA is an interfering RNA such as a shRNA, a microRNA, a guide RNA (gRNA) for use in combination with a Cas enzyme or similar enzyme for genome editing. In certain embodiments, the RNA is an antisense RNA capable of exon skipping such as a modified small nuclear RNA (snRNA) or a long non-coding RNA. The interfering RNA or microRNA may be used to regulate the expression of a target gene involved in muscle disease. The guide RNA in complex with a Cas enzyme or similar enzyme for genome editing may be used to modify the sequence of a target gene, in particular to correct the sequence of a mutated/deficient gene or to modify the expression of a target gene involved in a disease, in particular a neuromuscular disease. The antisense RNA capable of exon skipping is used in particular to correct a reading frame and restore expression of a deficient gene having a disrupted reading frame. In some embodiments, the RNA is a therapeutic RNA.

The genome-editing enzyme may be any enzyme or enzyme complex capable of modifying a target gene or target cellular pathway, in particular in muscle cells. For example, the genome-editing enzyme may modify the expression, sequence or regulation of the target gene or cellular pathway. In certain embodiments, the genome-editing enzyme is an engineered nuclease, such as with no limitations, a meganuclease, zinc finger nuclease (ZFN), transcription activator-like effector-based nuclease (TALENs), Cas enzyme from clustered regularly interspaced palindromic repeats (CRISPR)-Cas system and similar enzymes. The genome-editing enzyme, in particular an engineered nuclease such as Cas enzyme and similar enzymes, may be a functional nuclease which generates a double strand break (DSB) or single-stranded DNA break (nickase such as Cas9(D10A) in the target genomic locus and is used for site-specific genome editing applications, including with no limitations: gene correction, gene replacement, gene knock-in, gene knock-out, mutagenesis, chromosome translocation, chromosome deletion, and the like.

For site-specific genome editing applications, the genome-editing enzyme, in particular an engineered nuclease such as Cas enzyme and similar enzymes may be used in combination with a homologous recombination (HR) matrix or template (also named DNA donor template) which modifies the target genomic locus by double-strand break (DSB)-induced homologous recombination. In particular, the HR template may introduce a transgene of interest into the target genomic locus or repair a mutation in the target genomic locus, preferably in an abnormal or deficient gene causing a muscle or central nervous system (CNS) disorder, such as for example a neuromuscular disease.

Alternatively, the genome-editing enzyme, such as Cas enzyme and similar enzymes may be engineered to become nuclease-deficient and used as DNA-binding protein for various genome engineering applications such as with no limitation: transcriptional activation, transcriptional repression, epigenomic modification, genome imaging, DNA or RNA pull-down and the like.

### 7. Treatable Diseases and Conditions

The rAAV vector particle, cell and derived pharmaceutical composition of the invention may be used for treating diseases by, for example, gene therapy, in particular targeted gene therapy directed to muscle and/or CNS cells or tissue.

The cell and derived pharmaceutical composition of the invention may be used for treating diseases by, for example, cell therapy, in particular cell therapy directed to muscle and/or CNS cell or other target cells of interest.

As used herein, “gene therapy” includes a treatment of an individual which involves delivery of nucleic acid of interest into an individual's cells for the purpose of treating a disease. Delivery of the nucleic acid is generally achieved using a delivery vehicle, also known as a vector. The rAAV vector particle of the invention may be employed to deliver a gene to a patient's cells.

As used herein, “cell therapy” includes a process wherein cells stably transduced by a rAAV vector particle of the invention are delivered to the individual in need thereof by any appropriate means, such as, for example, by intravenous injection (infusion), or injection in the tissue of interest (implantation or transplantation).

In particular embodiments, cell therapy comprises collecting cells from the individual, transducing the individual's cells with the rAAV vector particle of the invention, and administering the stably transduced cells back to the patient.

Here, “cell” refers to an isolated cell, natural or artificial cellular aggregate, bioartificial cellular scaffold and bioartificial organ or tissue.

Gene therapy can be performed by gene transfer, gene editing, exon skipping, RNA-interference, trans-splicing or any other genetic modification of any coding or regulatory sequences in the cell, including those included in the nucleus, mitochondria or as commensal nucleic acid such as with no limitation viral sequences contained in cells.

In certain embodiments, the gene therapy provides a functional replacement gene for a deficient/abnormal gene, as used in replacement or additive gene therapy.

In certain embodiments, the gene therapy comprises gene or genome editing—e.g., to provide to a cell the necessary tools to correct the sequence or modify the expression or regulation of a deficient/abnormal gene so that a functional gene is expressed or an abnormal gene is suppressed (inactivated), as used in gene editing therapy.

In certain embodiments, the gene therapy comprises additive gene therapy, in which the gene of interest may be a functional version of a gene that is deficient or mutated in a patient, as is the case, for example, in a genetic disease. In such a case, the gene of interest will restore the expression of a functional gene. Thus, by gene editing or gene replacement a correct version of this gene is provided in target cells, in particular muscle and/or CNS cells or other target cells of affected patients, this may contribute to effective therapies against the disease.

In certain embodiments, the genome editing uses one or more gene(s) of interest, such as:
(A) a gene encoding a therapeutic RNA, such as an interfering RNA like a shRNA or a microRNA, a guide RNA (gRNA) for use in combination with a Cas enzyme or similar enzyme, or an antisense RNA capable of exon skipping such as a modified small nuclear RNA (snRNA); and
(B) a gene encoding a genome-editing enzyme as defined above such as an engineered nuclease like a meganuclease, zinc finger nuclease (ZFN), transcription activator-like effector-based nuclease (TALENs), Cas enzyme or similar enzymes; or a combination of such genes, and maybe also a fragment of a functional version of a gene for use as recombination template, as defined above.

In certain embodiments, gene therapy is used for treating various inherited (genetic) or acquired diseases or disorders affecting the structure or function of target tissue(s), in particular muscle(s) and/or the CNS, including skeletal or cardiac muscle(s), the brain or spinal cord. The diseases may be caused by trauma, infection, degeneration, structural or metabolic defects, tumors, autoimmune disorders, stroke or others. Non-limiting examples of diseases that can be treated by gene therapy include neuromuscular genetic disorders such as muscular genetic disorders; cancer; neurodegenerative diseases and auto-immune diseases.

In some embodiments, the target gene for gene therapy (additive gene therapy or gene editing) is a gene responsible for a neuromuscular disease.

Neuromuscular genetic disorders include in particular: Muscular dystrophies, Congenital muscular dystrophies, Congenital myopathies, Distal myopathies, Other myopathies, Myotonic syndromes, Ion Channel muscle diseases, Malignant hyperthermia, Metabolic myopathies, Hereditary Cardiomyopathies, Congenital myasthenic syndromes, Motor Neuron diseases, Hereditary paraplegia, Hereditary motor and sensory neuropathies and other neuromuscular disorders.

In some embodiments, the target gene for gene therapy (additive gene therapy or gene editing) is a gene responsible for a neuromuscular disease is Duchenne muscular dystrophy (DMD gene), Limb-girdle muscular dystrophies (LGMDs) (CAPN3, DYSF, FKRP, AN05 genes and others), Spinal muscular atrophy (SMN1 gene), myotubular myopathy (MTM1 gene), Pompe disease (GAA gene) and Glycogen storage disease III (GSD3) (AGL gene).

Dystrophinopathies are a spectrum of X-linked muscle diseases caused by pathogenic variants in DMD gene, which encodes the protein dystrophin. Dystrophinopathies comprises Duchenne muscular dystrophy (DMD), Becker muscular dystrophy (BMD) and DMD-associated dilated cardiomyopathy.

The Limb-girdle muscular dystrophies (LGMDs) are a group of disorders that are clinically similar to DMD but occur in both sexes as a result of autosomal recessive and autosomal dominant inheritance. Limb-girdle dystrophies are caused by mutation of genes that encode sarcoglycans and other proteins associated with the muscle cell membrane, which interact with dystrophin. The term LGMD1 refers to genetic types showing dominant inheritance (autosomal dominant), whereas LGMD2 refers to types with autosomal recessive inheritance. Pathogenic variants at more than 50 loci have been reported (LGMD1A to LGMD1G; LGMD2A to LGMD2W). Calpainopathy (LGMD2A) is caused by mutation of the gene CAPN3 with more than 450 pathogenic variants described. Contributing genes to LGMD phenotype include: anoctamin 5 (AN05), blood vessel epicardial substance (BVES), calpain 3 (CAPN3), caveolin 3 (CAV3), CDP-L-ribitol pyrophosphorylase A (CRPPA), dystroglycan 1 (DAG1), desmin (DES), DnaJ heat shock protein family (Hsp40) homolog, subfamily B, member 6 (DNAJB6), dysferlin (DYSF), fukutin related protein (FKRP), fukutin (FKT), GDP-mannose pyrophosphorylase B (GMPPB), heterogeneous nuclear ribonucleoprotein D like (FINRNPDL), LIM zinc finger domain containing 2 (LIMS2), lain A:C (LMNA), myotilin (MYOT), plectin (PLEC), protein O-glucosyltransferase 1 (PLOGLUTI), protein O-linked mannose N-acetylglucosaminyltransferase 1 (beta 1,2-) (POMGNT1), protein O-mannose kinase (POMK), protein O-mannosyltransferase 1 (POMT1), protein O-mannosyltransferase 2 (POMT2), sarcoglycan alpha (SGCA), sarcoglycan beta (SGCB), sarcoglycan delta (SGCD), sarcoglycan gamma (SGCG), titin-cap (TCAP), transportin 3 (TNPO3), torsin 1A interacting protein (TOR1AIP1), trafficking protein particle complex 11 (TRAPPC11), tripartite motif containing 32 (TRIM 32) and titin (TTN). Major contributing genes to LGMD phenotype include CAPN3, DYSF, FKRP and AN05 (Babi Ramesh Reddy Nallamilli et al., Annals of Clinical and Translational Neurology, 2018, 5, 1574-1587).

Spinal muscular atrophy is a genetic disorder caused by mutations in the Survival Motor Neuron 1 (SMN1) gene which is characterized by weakness and wasting (atrophy) in muscles used for movement.

X-linked myotubular myopathy is a genetic disorder caused by mutations in the myotubularin (MTM1) gene which affects muscles used for movement (skeletal muscles) and occurs almost exclusively in males. This condition is characterized by muscle weakness (myopathy) and decreased muscle tone (hypotonia).

Pompe disease is a genetic disorder caused by mutations in the acid alpha-glucosidase (GAA) gene. Mutations in the GAA gene prevent acid alpha-glucosidase from breaking down glycogen effectively, which allows this sugar to build up to toxic levels in lysosomes. This buildup damages organs and tissues throughout the body, particularly the muscles, leading to the progressive signs and symptoms of Pompe disease.

Thus, in certain embodiments, the (genetic) disease or disorder is Pompe disease or GAA deficiency, and the GOI encodes α-glucosidase alglucosidase alfa.

In certain embodiments, the (genetic) disease or disorder is Fabry disease or a deficiency of α-galactosidase A (α-Gal A), and the GOI encodes α-galactosidase A.

In certain embodiments, the (genetic) disease or disorder is Gaucher disease or beta-glucocerebrosidase deficiency, and the GOI encodes beta-glucocerebrosidase.

In certain embodiments, the (genetic) disease or disorder is Hunter syndrome or MPS-II, and the GOI encodes lysosomal enzyme iduronate-2-sulfatase.

In certain embodiments, the (genetic) disease or disorder is hypophosphatasia (HPP), such as perinatal/infantile- and juvenile-onset HPP, and the GOI encodes asfotase alfa.

In certain embodiments, the (genetic) disease or disorder is lysosomal acid lipase deficiency (LAL-D), and the GOI encodes lysosomal acid lipase (LAL) or sebelipase alfa.

In certain embodiments, the (genetic) disease or disorder is Hurler syndrome/Scheie syndrome, and the GOI encodes Iduronidase (IDUA).

In certain embodiments, the (genetic) disease or disorder is Maroteaux-Lamy syndrome, and the GOI encodes arylsulfatase B (ARSB).

In certain embodiments, the (genetic) disease or disorder is sphingomyelinase deficiency (ASMD), and the GOI encodes SMPD1.

In certain embodiments, the (genetic) disease or disorder is Type1 Diabetes, Type2 Diabetes and hyperglycemia, and the GOI encodes Insulin.

Glycogen storage disease III (GSD3) is an autosomal recessive metabolic disorder caused by homozygous or compound heterozygous mutation in the Amylo-Alpha-1, 6-Glucosidase, 4-Alpha-Glucanotransferase (AGL) gene which encodes the glycogen debrancher enzyme and associated with an accumulation of abnormal glycogen with short outer chains. Clinically, patients with GSD III present in infancy or early childhood with hepatomegaly, hypoglycemia, and growth retardation. Muscle weakness in those with Ilia is minimal in childhood but can become more severe in adults; some patients develop cardiomyopathy.

In certain embodiments, the (genetic) disease or disorder is in CNS, and has a defect in one or more of the following gene (associated with the condition in parenthesis) that can be corrected by the GOI: CLN3 (Neuro Ceroid-Lipofuscinosis); FUCA1 (Fucosidosis); GAN (Giant Axonal Neuropathy); GALC (Globoid cell leukodystrophy); GALC (Mucolipidiosis Type IV); MCOLN1 (Mucolipidiosis Type IV); PPT1 (Neuronal Ceroid Lipofusinoses); SMPD1 (Niemann-Pick Disease); HEXB (Sandhoff Disease); SGSH (Sanfilippo syndrome); HEXA (Tay-Sachs Disease); NEU1 (Sialidosis); SUMF1 (Multiple Sulfatase Deficiency); GAT1/SLCA1 (Childhood Epilepsy); CMT FIG. 4 (Peripheral Neuropathy); CLN5 (Neuronal Ceroid Lipofusinoses); AGA (Aspartylglycosaminuria); GDNF (Parkinsons Disease/Symptoms); GLB1 (GM1-Gangliosidosis); PMP22/MFN2 (Charcot-Marie-Tooth Type 1A); MECP2 (Retts Syndrome); LAMP2 (Dannon Disease); NAGLU (Mucopolysaccharidoses); GUSB (Sly Syndrome); SLC19A3 (Biotin basal ganglia disease); PLP1 (Pelizaeus-Merzbacher disease); TPP1/CLN2 (Neuronal Ceroid Lipofusinoses); ACY2/ASPA (Canavan Disease); MANBA (Beta-Mannosidosis); CTNS (Cystinosis); GNS (Mucopolysaccharidoses); HGSNAT (Mucopolysaccharidoses); SLC17A5 (Salla Disease); CLN6 (Jansky-Bielschowsky disease); CLN8 (Neuronal ceroid lipofuscinoses); and GM2 (gangliosidosis).

Replacement or additive gene therapy may be used to treat cancer, in particular rhabdomyosarcomas. Genes of interest in cancer could regulate the cell cycle or the metabolism and migration of the tumor cells, or induce tumor cell death. For instance, inducible caspase-9 could be expressed in muscle cells to trigger cell death, preferably in combination therapy to elicit durable anti-tumor immune responses.

Gene editing may be used to modify gene expression in target cells, in particular muscle and/or CNS cells, in the case of auto-immunity or cancer, or to perturb the cycle of viruses in such cells. In such cases, the gene of interest may be chosen from those encoding guide RNA (gRNA), site-specific endonucleases (TALEN, meganucleases, zinc finger nucleases, Cas nuclease), DNA templates and RNAi components, such as shRNA and microRNA. Tools such as CRISPR/Cas9 may be used for this purpose.

In some embodiments, gene therapy is used for treating diseases affecting other tissues, by expression of a therapeutic gene in target tissue, in particular, muscle and/or CNS tissue. This is useful to avoid expression of the therapeutic gene in the liver, in particular in patients having a concurrent hepatic disorder such as hepatitis. The therapeutic gene encodes preferably a therapeutic protein, peptide or antibody which is secreted from the muscle cells into the blood stream where it can be delivered to other target tissues such as for example the liver. Examples of therapeutic genes include with no limitation: Factor VIII, Factor IX and GAA genes.

In the various embodiments of the present invention, the pharmaceutical composition comprises a therapeutically effective amount of rAAV vector particle or cell.

In the context of the invention, a “therapeutically effective amount” refers to a dose sufficient for reversing, alleviating or inhibiting the progress of the disorder or condition to which such term applies, or reversing, alleviating or inhibiting the progress of one or more symptoms of the disorder or condition to which such term applies.

The term “effective dose” or “effective dosage” is defined as an amount sufficient to achieve, or at least partially achieve, the desired effect.

The effective dose is determined and adjusted depending on factors such as the composition used, the route of administration, the physical characteristics of the individual under consideration such as sex, age and weight, concurrent medication, and other factors, that those skilled in the medical arts will recognize.

In a related aspect, the invention provides a method for treating a disease by expression of a therapeutic gene in a target tissue, in particular muscle and/or CNS tissue, comprising: administering to a patient a therapeutically effective amount of the pharmaceutical composition of the invention as described above.

Another aspect of the invention relates to the rAAV vector particle, cell, pharmaceutical composition according to the present disclosure as a medicament, in particular for use in the treatment of a muscle or CNS disorder according to the present disclosure, in particular neuromuscular genetic disease.

The invention provides also a method for treating a muscle or CNS disorder, comprising: administering to a patient a therapeutically effective amount of the pharmaceutical composition as described above, comprising at least an active agent selected from an AAV vector particle or a cell of the invention, and a pharmaceutically acceptable carrier.

A further aspect of the invention relates to the use of a rAAV vector particle, cell according to the present disclosure in the manufacture of a medicament for the treatment of a muscle or CNS disorder, in particular neuromuscular genetic disease.

Another aspect of the invention relates to the use of a rAAV vector particle or a cell of the present disclosure for the treatment of a muscle or CNS disorder according to the present disclosure, in particular neuromuscular genetic disease.

A further aspect of the invention relates to a pharmaceutical composition for use in treating a muscle or CNS disorder according to the present disclosure, in particular neuromuscular genetic disease, comprising an AAV vector particle or a cell of the present disclosure as an active component.

A further aspect of the invention relates to a pharmaceutical comprising an AAV vector particle or a cell of the present disclosure for treating a muscle or CNS disorder according to the present disclosure, in particular neuromuscular genetic disease.

As used herein, the term “patient” or “individual” includes human and other mammalian subjects that receive either prophylactic or therapeutic treatment. In certain embodiments, a patient or individual according to the invention is a human.

“Treatment,” or “treating,” as used herein, includes application or administration of a therapeutic agent or combination of therapeutic agents to a patient, or application or administration of said therapeutic agents to an isolated tissue or cell line from a patient, who has a disease, in particular a muscle or CNS disorder with the purpose to cure, heal, alleviate, relieve, alter, remedy, ameliorate, improve or affect the disease, or any symptom of the disease. In particular, the terms “treat” or “treatment” refers to reducing or alleviating at least one adverse clinical symptom associated with the disease.

In certain embodiments, the term “treatment” or “treating” is also used herein in the context of administering the therapeutic agents prophylactically.

In certain other embodiments, the term “treatment” or “treating” excluded administering the therapeutic agents prophylactically.

For any of the treatment methods, use, or pharmaceutical compositions for use thereof, the pharmaceutical composition of the present invention is generally administered according to known procedures, at dosages and for periods of time effective to induce a therapeutic effect in the patient. The pharmaceutical composition may be administered by any convenient route, such as in a non-limiting manner by infusion or bolus injection, by absorption through epithelial or mucocutaneous linings (e.g., oral mucosa, rectal and intestinal mucosa, etc.). The administration can be systemic, local or systemic combined with local; systemic includes parenteral and oral, and local includes local and loco-regional. Systemic administration may be parenteral such as subcutaneous (s.c.), intramuscular (i.m.), intravascular such as intravenous (i.v.) or intraarterial; intraperitoneal (i.p.); intradermal (i.d.), epidural or else.

The parenteral administration is advantageously by injection or perfusion. Local administration is preferably intracerebral, intracerebroventricular, intracisternal, and/or intrathecal administration. The administration may be for example by injection or perfusion. In some embodiments, the administration is parenteral, e.g., intravascular, such as intravenous (i.v.) or intraarterial. In some other embodiments, the administration is intracerebral, intracisternal, intracerebroventricular, and/or intrathecal administration, alone or combined with parenteral administration, such as intravascular administration. In some other embodiments, the administration is parenteral, such as intravascular alone or combined with intracerebral, intracerebroventricular, intracisternal, and/or intrathecal administration.

### 8. Combination Therapy

Combination therapies are also contemplated by the invention. Combination as used herein includes both simultaneous treatment or sequential treatments. Combinations of methods of the invention with standard medical treatments are specifically contemplated, as are combinations with novel therapies.

All publications and patents mentioned herein are hereby incorporated by reference in their entirety as if each individual publication or patent was specifically and individually indicated to be incorporated by reference. In case of conflict, the present application, including any definitions herein, will control.

The various embodiments of the present disclosure can be combined with each other and the present disclosure encompasses the various combinations of embodiments of the present disclosure.

The invention is further described in the following Examples, which do not limit the scope of the invention described in the claims.

### EXAMPLES

### Example 1 Identifying Clinically Validated AAV Capsids with Enhanced Nuclear Localization

This example demonstrates that certain clinically validated AAV capsids (e.g., those that have been demonstrated to be safe or suitable for or in clinical trials) have enhanced nuclear localization compared to certain other clinically validated AAV capsids.

Four specific clinically validated AAV capsids were chosen for this experiment: AAV5 and AAV6 (both validated for AAV-mediated treatment for Hemophilia A), AAV8 (for AAV-mediated treatment for myotubular myopathy), and AAV9 (for AAV-mediated treatment for Duchenne Muscular Dystrophy r DMD).

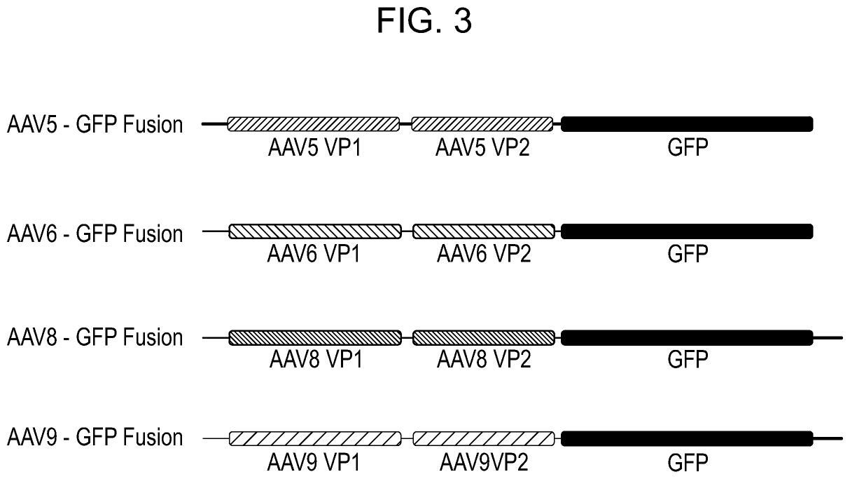

The VP1-N and VP2-N regions of these AAV capsid polypeptides were fused to a reporter protein—GFP in this case (see FIG. 3), and the constructs encoding the fusions were verified via agarose gel electrophoresis (FIG. 4). A NLS-GFP (replacing the AAV capsid portion with a nuclear localization signal (NLS) was used as a positive control.

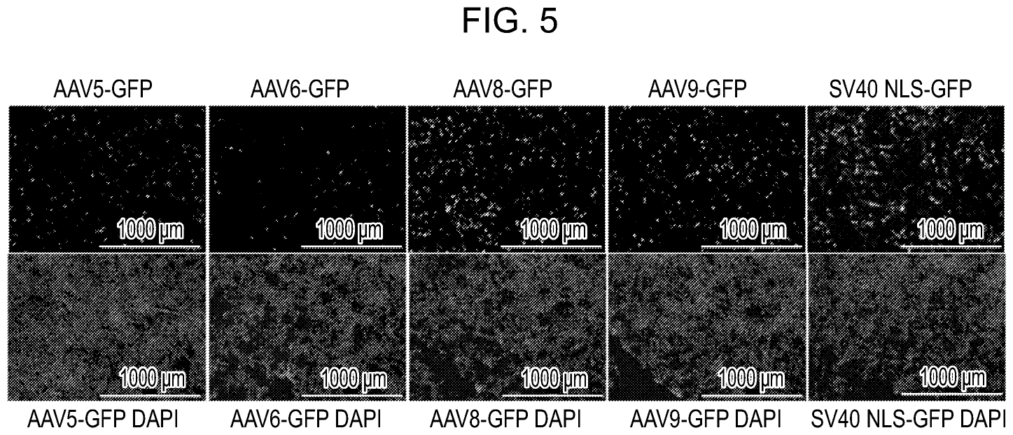

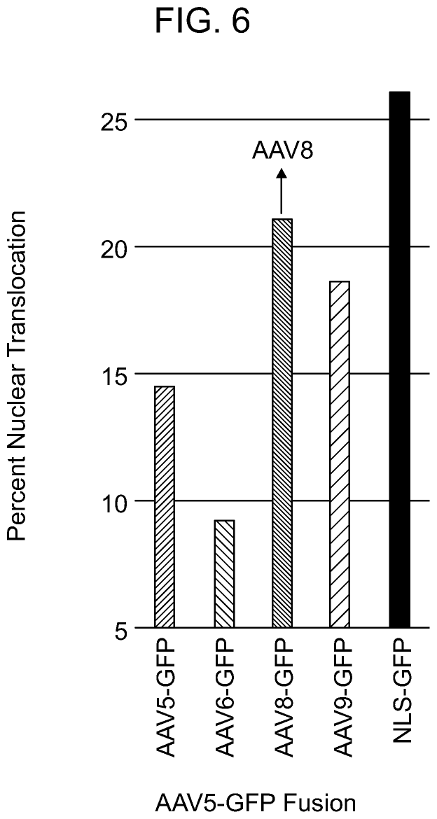

Transfection of muscle cells using these constructs showed that two of the AAV fusions—AAV8 and AAV9 fusions with GFP, were more prominently expressed in the muscle cells compared to the other two AAV fusions (AAV5 and AAV6). See FIG. 5. Quantitative analysis based on the signal intensities confirmed that the AAV8 fusion exhibited the highest expression, with the AAV9 fusion close behind. See FIG. 6.

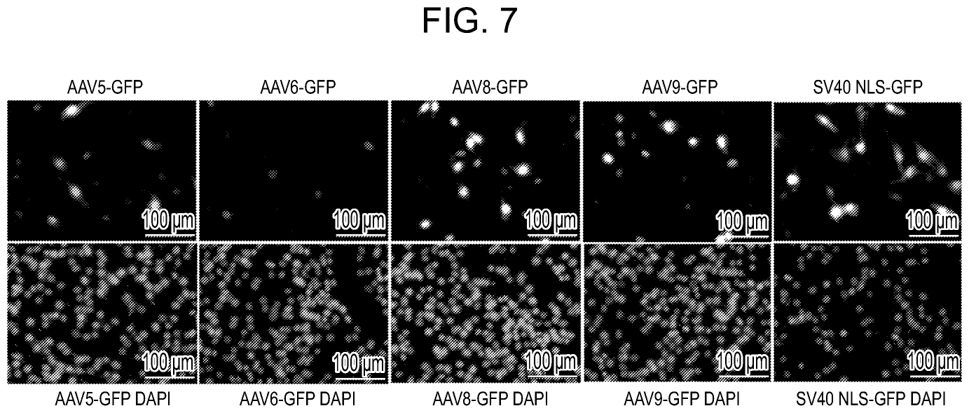

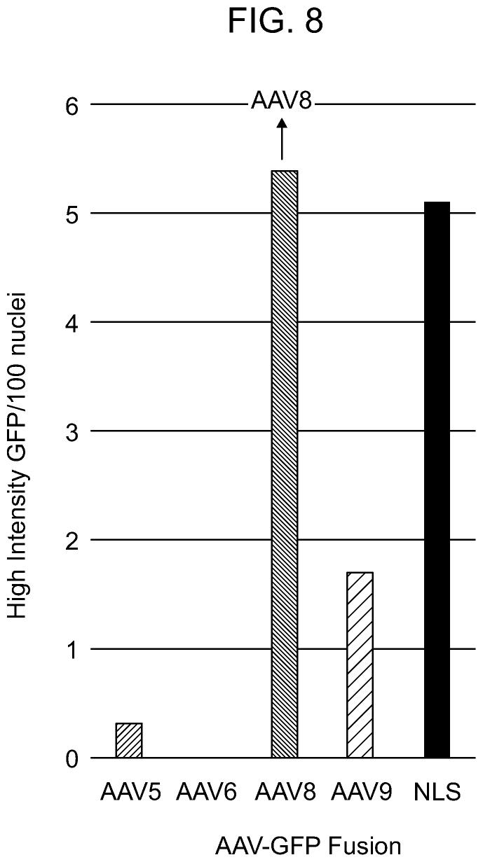

Similarly, nuclear translocation intensity data in FIGS. 7 and 8 confirmed the same finding. Thus, AAV8—more specifically the VP1-N and VP2-N regions of the AAV8 VP1 capsid—was verified as containing a superior nuclear localization signal compared to the other clinically validated AAV capsids, and is thus chosen for further construction of the hybrid capsids of the invention.

### Example 2 Engineered/Hybrid AAV9-8 Capsid with Enhanced Nuclear Localization

Based on the results in Example 1, one exemplary (non-limiting) engineered hybrid capsid of the invention was constructed with the following polynucleotide and polypeptide sequences:

Engineered AAV 9-8 (also known as AAV8X-AAV9) capsid polynucleotide
sequence:
(SEQ ID NO: 1)
ATGGCTGCCGATGGTTATCTTCCAGATTGGCTCGAGGACAACCTCTCTGAGGGCATTCGCGA
GTGGTGGGCGCTGAAACCTGGAGCCCCGAAGCCCAAAGCCAACCAGCAAAAGCAGGACGACG
GCCGGGGTCTGGTGCTTCCTGGCTACAAGTACCTCGGACCCTTCAACGGACTCGACAAGGGG
GAGCCCGTCAACGCGGCGGACGCAGCGGCCCTCGAGCACGACAAGGCCTACGACCAGCAGCT
GCAGGCGGGTGACAATCCGTACCTGCGGTATAACCACGCCGACGCCGAGTTTCAGGAGCGTC
TGCAAGAAGATACGTCTTTTGGGGGCAACCTCGGGCGAGCAGTCTTCCAGGCCAAGAAGCGG
GTTCTCGAACCTCTCGGTCTGGTTGAGGAAGGCGCTAAGACGGCTCCTGGAAAGAAGAGACC
GGTAGAGCCATCACCCCAGCGTTCTCCAGACTCCTCTACGGGCATCGGCAAGAAAGGCCAAC
AGCCCGCCAGAAAAAGACTCAATTTTGGTCAGACTGGCGACTCAGAGTCAGTTCCAGACCCT
CAACCTCTCGGAGAACCTCCAGCAGCGCCCTCTGGTGTGGGACCTAATACAATGGCTGCAGG
CGGTGGCGCACCAATGGCAGACAATAACGAAGGCGCCGACGGAGTGGGTAGTTCCTCGGGAA
ATTGGCATTGCGATTCCCAATGGCTGGGGGACAGAGTCATCACCACCAGCACCCGAACCTGG
GCCCTGCCCACCTACAACAATCACCTCTACAAGCAAATCTCCAACAGCACATCTGGAGGATC
TTCAAATGACAACGCCTACTTCGGCTACAGCACCCCCTGGGGGTATTTTGACTTCAACAGAT
TCCACTGCCACTTCTCACCACGTGACTGGCAGCGACTCATCAACAACAACTGGGGATTCCGG
CCTAAGCGACTCAACTTCAAGCTCTTCAACATTCAGGTCAAAGAGGTTACGGACAACAATGG
AGTCAAGACCATCGCCAATAACCTTACCAGCACGGTCCAGGTCTTCACGGACTCAGACTATC
AGCTCCCGTACGTGCTCGGGTCGGCTCACGAGGGCTGCCTCCCGCCGTTCCCAGCGGACGTT
TTCATGATTCCTCAGTACGGGTATCTGACGCTTAATGATGGAAGCCAGGCCGTGGGTCGTTC
GTCCTTTTACTGCCTGGAATATTTCCCGTCGCAAATGCTAAGAACGGGTAACAACTTCCAGT
TCAGCTACGAGTTTGAGAACGTACCTTTCCATAGCAGCTACGCTCACAGCCAAAGCCTGGAC
CGACTAATGAATCCACTCATCGACCAATACTTGTACTATCTCTCAAAGACTATTAACGGTTC
TGGACAGAATCAACAAACGCTAAAATTCAGTGTGGCCGGACCCAGCAACATGGCTGTCCAGG
GAAGAAACTACATACCTGGACCCAGCTACCGACAACAACGTGTCTCAACCACTGTGACTCAA
AACAACAACAGCGAATTTGCTTGGCCTGGAGCTTCTTCTTGGGCTCTCAATGGACGTAATAG
CTTGATGAATCCTGGACCTGCTATGGCCAGCCACAAAGAAGGAGAGGACCGTTTCTTTCCTT
TGTCTGGATCTTTAATTTTTGGCAAACAAGGAACTGGAAGAGACAACGTGGATGCGGACAAA
GTCATGATAACCAACGAAGAAGAAATTAAAACTACTAACCCGGTAGCAACGGAGTCCTATGG
ACAAGTGGCCACAAACCACCAGAGTGCCCAAGCACAGGCGCAGACCGGCTGGGTTCAAAACC
AAGGAATACTTCCGGGTATGGTTTGGCAGGACAGAGATGTGTACCTGCAAGGACCCATTTGG
GCCAAAATTCCTCACACGGACGGCAACTTTCACCCTTCTCCGCTGATGGGAGGGTTTGGAAT
GAAGCACCCGCCTCCTCAGATCCTCATCAAAAACACACCTGTACCTGCGGATCCTCCAACGG
CCTTCAACAAGGACAAGCTGAACTCTTTCATCACCCAGTATTCTACTGGCCAAGTCAGCGTG
GAGATCGAGTGGGAGCTGCAGAAGGAAAACAGCAAGCGCTGGAACCCGGAGATCCAGTACAC
TTCCAACTATTACAAGTCTAATAATGTTGAATTTGCTGTTAATACTGAAGGTGTATATAGTG
AACCCCGCCCCATTGGCACCAGATACCTGACTCGTAATCTGTAA
Engineered AAV 9-8 (also known as AAV8X-AAV9) capsid polypeptide sequence:
(SEQ ID NO: 2)
MAADGYLPDWLEDNLSEGIREWWALKPGAPKPKANQQKQDDGRGLVLPGYKYLGPFNGLDKG
EPVNAADAAALEHDKAYDQQLQAGDNPYLRYNHADAEFQERLQEDTSFGGNLGRAVFQAKKR
VLEPLGLVEEGAKTAPGKKRPVEPSPQRSPDSSTGIGKKGQQPARKRLNFGQTGDSESVPDP
QPLGEPPAAPSGVGPNTMAAGGGAPMADNNEGADGVGSSSGNWHCDSQWLGDRVITTSTRTW
ALPTYNNHLYKQISNSTSGGSSNDNAYFGYSTPWGYFDFNRFHCHFSPRDWQRLINNNWGFR
PKRLNFKLFNIQVKEVTDNNGVKTIANNLTSTVQVFTDSDYQLPYVLGSAHEGCLPPFPADV
FMIPQYGYLTLNDGSQAVGRSSFYCLEYFPSQMLRTGNNFQFSYEFENVPFHSSYAHSQSLD
RLMNPLIDQYLYYLSKTINGSGQNQQTLKFSVAGPSNMAVQGRNYIPGPSYRQQRVSTTVTQ
NNNSEFAWPGASSWALNGRNSLMNPGPAMASHKEGEDRFFPLSGSLIFGKQGTGRDNVDADK
VMITNEEEIKTTNPVATESYGQVATNHQSAQAQAQTGWVQNQGILPGMVWQDRDVYLQGPIW
AKIPHTDGNFHPSPLMGGFGMKHPPPQILIKNTPVPADPPTAFNKDKLNSFITQYSTGQVSV
EIEWELQKENSKRWNPEIQYTSNYYKSNNVEFAVNTEGVYSEPRPIGTRYLTRNL

In the above AAV9-8 polypeptide sequence having a total of 737 residues, residues 1-220 are identical to residues 1-220 of AAV8 VP1, and residues 221-737 are identical to residues 220-736 of AAV9 VP1. Further, residues 213-220 of AAV9-8 are common in both AAV8 and AAV9 VP1 polypeptides.

Thus, the enhanced nuclear localization signal in the AAV8 VP1 polypeptide resides within the VP1-N domain (roughly the first 137 residues of AAV8 VP1), the VP2-N domain (roughly the next 65 residues of AAV8 VP1), and the most N-terminal portion of the VP3 domain (roughly about the most N-terminal 10-20 residues of AAV8 VP3 domain).

The coding sequence for this representative hybrid capsid VP1 polypeptide was cloned into an AAV repcap plasmid, which also encodes an AAV2 Rep coding sequence, and which can be used in, for example, the triple transfection method to facilitate AAV production in conjunction with a construct encoding the GOI flanked by AAV ITR sequences (such as AAV2 ITR sequences).

The viral particles so produced would comprise a vector genome with the GOI flanked by AAV2 ITR sequences, encapsidated with the subject AAV9-8 hybrid capsid, which is expected to show enhanced nuclear translocation in a target cell, such as muscle cells or neuronal cells.

1. A polynucleotide encoding an engineered adeno-associated virus (AAV) capsid comprising a viral protein 1 (VP1), a viral protein 2 (VP2), and a viral protein 3 (VP3), wherein said VP1 comprises:
(a) a first portion from a first AAV capsid (e.g., AAV8) VP1, wherein said first portion comprises a nuclear localization sequence (NLS) of said first AAV capsid (e.g., AAV8) VP1; and,
(b) a second portion from a second AAV capsid (e.g., AAV9) VP1, wherein said second portion comprises a tropism region associated with a tropism of the second capsid (e.g., AAV9) VP1 for a target cell;
wherein the first and the second AAV capsids have different tropism or serotype.

2. The polynucleotide of claim 1, wherein said NLS, or said first portion comprising said NLS, when present in a fusion with a reporter (e.g., a fluorescent protein such as GFP), directs the subcellular localization of the fusion to the nucleus of a cell (e.g., muscle cell or myoblast) expressing the fusion.

3. The polynucleotide of claim 1 or 2, wherein said first AAV capsid is from a clinically validated AAV serotype (such as AAV5, AAV6, AAV8, or AAV9).

4. The polynucleotide of any one of claims 1-3, wherein the first portion comprises the VP1-N region and the VP2-N region of the first AAV capsid VP1, and wherein the tropism region is within the VP3 region of the second AAV capsid VP1.

5. The polynucleotide of any one of claims 1-4, wherein the first portion (of the first AAV capsid VP1) substantially excludes the VP3 region of said first AAV capsid VP1, and the second portion (of the second AAV capsid VP1) comprises, consists essentially of, or consists of the VP3 region of said second AAV capsid VP1.

6. The polynucleotide of any one of claims 1-5, wherein said first AAV capsid VP1 is from AAV8, and said second AAV capsid VP1 is from AAV9.

7. The polynucleotide of any one of claims 1-6, encoding the N-terminal 212-220 residues of AAV8 VP1.

8. The polynucleotide of any one of claims 1-7, encoding the C-terminal 517-525 residues (e.g., residues 220-736 or residues 212-736) of AAV9 VP1.

9. The polynucleotide of any one of claims 1-8, wherein the engineered AAV capsid comprises, consists essentially of, or consists of the polypeptide sequence of SEQ ID NO: 2, or a variant at least 90%, 91%, 92%, 93%, 94%, 95%, 96%, 97%, 98%, 99%, 99.2%, 99.4%, 99.6, 99.8%, or 99.9% identical thereto.

10. The polynucleotide of claim 9, which is codon optimized for expression in a mammalian cell (such as HEK293T cells) or an insect cell (such as Sf9).

11. The polynucleotide of any one of claims 1-10, comprising, consisting essentially of, or consisting of the polynucleotide sequence of SEQ ID NO: 1.

12. A vector comprising the polynucleotide of any one of claims 1-11.

13. The vector of claim 12, comprising a promoter operably linked to a transcription cassette comprising the polynucleotide of any one of claims 1-11.

14. The vector of claim 13, further comprising a coding sequence for an AAV rep.

15. An engineered adeno-associated virus (AAV) capsid VP1, VP2, and/or VP3, encoded by the polynucleotide of any one of claims 1-11.

16. A host cell comprising the vector of any one of claims 12-14.

17. The host cell of claim 16, further comprising a gene-of-interest (GOI) flanked by an AAV ITR sequence, capable of being encapsidated in the capsid of claim 16.

18. The host cell of claim 17, wherein the GOI is operably linked to a promoter.

19. The host cell of claim 18, wherein the promoter comprises:
(1) a constitutively active eukaryotic promoter,
(2) a muscle-specific promoter, a CNS-specific promoter, and/or
(3) a synthetic promoter.

20. The host cell of any one of claims 16-19, wherein the GOI further encodes a 5′-UTR region, a 3′-UTR region, and/or a polyA signal.

21. An AAV viral particle comprising the AAV capsid of claim 15.

22. The AAV viral particle of claim 21, comprising an AAV vector genome comprising a GOI flanked by an AAV ITR sequence, wherein the GOI is operably linked to a promoter,
optionally, the promoter comprises:
(1) a constitutively active eukaryotic promoter,
(2) a muscle-specific promoter, a CNS-specific Promoter, and/or
(3) a synthetic promoter, and/or,
optionally, the GOI further encodes a 5′-UTR region, a 3′-UTR region, and/or a polyA signal.

23. The AAV viral particle of claim 22, wherein the GOI encodes a functional protein (such as a therapeutic protein), a functional nucleic acid, and/or an antibody or a portion thereof (such as a heavy chain and/or a light chain of the antibody).

24. The AAV viral particle of claim 23, wherein the functional protein comprises a CRISPR/Cas effector enzyme (such as Cas9, Cas12, or Cas13), and/or wherein the functional nucleic acid is a single guide RNA (sgRNA), siRNA, miRNA, shRNA, antisense RNA, ribozyme, or aptamer.

25. The AAV viral particle of claim 23, wherein the functional protein comprises α-glucosidase alglucosidase alfa, α-galactosidase A (α-Gal A), beta-glucocerebrosidase, lysosomal enzyme iduronate-2-sulfatase, asfotase alfa, lysosomal acid lipase (LAL), sebelipase alfa, Iduronidase (IDUA), arylsulfatase B (ARSB), SMPD1, or Insulin.

26. The AAV viral particle of claim 23, wherein the functional protein comprises or is encoded by: CLN3 (associated with Neuro Ceroid-Lipofuscinosis); FUCA1 (associated with Fucosidosis); GAN (associated with Giant Axonal Neuropathy); GALC (associated with Globoid cell leukodystrophy); GALC (associated with Mucolipidiosis Type IV); MCOLN1 (associated with Mucolipidiosis Type IV); PPT1 (associated with Neuronal Ceroid Lipofusinoses); SMPD1 (associated with Niemann-Pick Disease); HEXB (associated with Sandhoff Disease); SGSH (associated with Sanfilippo syndrome); HEXA (associated with Tay-Sachs Disease); NEU1 (associated with Sialidosis); SUMF1 (associated with Multiple Sulfatase Deficiency); GAT1/SLCA1 (associated with Childhood Epilepsy); CMT FIG. 4 associated with (Peripheral Neuropathy); CLN5 (associated with Neuronal Ceroid Lipofusinoses); AGA (associated with Aspartylglycosaminuria); GDNF (associated with Parkinsons Disease/Symptoms); GLB1 (associated with GM1-Gangliosidosis); PMP22/MFN2 (associated with Charcot-Marie-Tooth Type 1A); MECP2 (associated with Retts Syndrome); LAMP2 (associated with Dannon Disease); NAGLU (associated with Mucopolysaccharidoses); GUSB (associated with Sly Syndrome); SLC19A3 (associated with Biotin basal ganglia disease); PLP1 (associated with Pelizaeus-Merzbacher disease); TPP1/CLN2 (associated with Neuronal Ceroid Lipofusinoses); ACY2/ASPA (associated with Canavan Disease); MANBA (associated with Beta-Mannosidosis); CTNS (associated with Cystinosis); GNS (associated with Mucopolysaccharidoses); HGSNAT (associated with Mucopolysaccharidoses); SLC17A5 (associated with Salla Disease); CLN6 (associated with Jansky-Bielschowsky disease); CLN8 (associated with Neuronal ceroid lipofuscinoses); GM2 (associated with gangliosidosis).

27. A pharmaceutical composition comprising the engineered AAV capsid VP1 of any one of claims 1-11, or the AAV viral particle of any one of claims 21-26, and a pharmaceutically acceptable carrier or excipient.

28. A method of treating a (genetic) disease or disorder in a subject, the method comprising introducing the AAV viral particle of any one of claims 21-26, or the pharmaceutical composition of claim 27, into the subject.

29. The method of claim 28, wherein the (genetic) disease or disorder is:
(1) Pompe disease or GAA deficiency, and the GOI encodes α-glucosidase alglucosidase alfa;
(2) Fabry disease or a deficiency of α-galactosidase A (α-Gal A), and the GOI encodes α-galactosidase A;
(3) Gaucher disease or beta-glucocerebrosidase deficiency, and the GOI encodes beta-glucocerebrosidase;
(4) Hunter syndrome or MPS-II, and the GOI encodes lysosomal enzyme iduronate-2-sulfatase;
(5) hypophosphatasia (HPP), such as perinatal/infantile- and juvenile-onset HPP, and the GOI encodes asfotase alfa;
(6) lysosomal acid lipase deficiency (LAL-D), and the GOI encodes lysosomal acid lipase (LAL) or sebelipase alfa;
(7) Hurler syndrome/Scheie syndrome, and the GOI encodes Iduronidase (IDUA);
(8) Maroteaux-Lamy syndrome, and the GOI encodes arylsulfatase B (ARSB);
(9) sphingomyelinase deficiency (ASMD), and the GOI encodes SMPD1; or,
(10) Type1 Diabetes, Type2 Diabetes and hyperglycemia, and the GOI encodes Insulin.

30. The method of claim 28, wherein the (genetic) disease or disorder is: Neuro Ceroid-Lipofuscinosis; Fucosidosis; Giant Axonal Neuropathy; Globoid cell leukodystroph; Mucolipidiosis Type IV; Mucolipidiosis Type IV; Neuronal Ceroid Lipofusinoses; Niemann-Pick Disease; Sandhoff Disease; Sanfilippo syndrome; Tay-Sachs Disease; Sialidosis; Multiple Sulfatase Deficiency; Childhood Epilepsy; Peripheral Neuropathy; Neuronal Ceroid Lipofusinoses; Aspartylglycosaminuria; Parkinsons Disease/Symptoms; GM1-Gangliosidosis; Charcot-Marie-Tooth Type 1A; Retts Syndrome; Dannon Disease; Mucopolysaccharidoses; Sly Syndrome; Biotin basal ganglia disease; Pelizaeus-Merzbacher disease; Canavan Disease; Beta-Mannosidosis; Cystinosis; Mucopolysaccharidoses; Mucopolysaccharidoses; Salla Disease; Jansky-Bielschowsky disease; Neuronal ceroid lipofuscinoses; or GM2 gangliosidosis.

31. A method of producing the AAV viral particle of claim any one of 21-26, comprising introducing the polynucleotide of any one of claims 1-6 into a packaging cell line that constitutively or inducibly expresses said polynucleotide and an AAV cap protein.
